(19) United States
(12) Patent Application Publication
(10) Pub. No.: US 2007/0265256 A1
(43) Pub. Date: Nov. 15, 2007

(54) Phosphodiesterase 10 inhibitors

(76) Inventors: Mark P. Arrington, Westwood, NJ (US); Ruiping Liu, Huntington, NY (US); Allen T. Hopper, Mahwa, NJ (US); Richard D. Conticello, Cortlandt Manor, NY (US); Truc M. Nguyen, New York, NY (US); Carla Maria Gauss, San Diego, CA (US); Roland Burli, Pasadena, CA (US); Stephen A. Hitchcock, Westlake Village, CA (US); Essa Hu, Camarillo, CA (US); Roxanne Kunz, Santa Monica, CA (US); Thomas Nixey, Newbury Park, CA (US); Shannon Rumfelt, Camarillo, CA (US); Nign Chen, Thousand Oaks, CA (US)

Correspondence Address: JONES DAY, 222 EAST 41ST ST, NEW YORK, NY, 10017, US

(21) Appl. No.: 11/708,866

(22) Filed: Feb. 20, 2007

Related U.S. Application Data
(60) Provisional application No. 60/774,550, filed on Feb. 21, 2006.

(51) Int. Cl. A61K 31/535; A61K 31/50; A61P 25/18; C07D 237/26; C07D 413/00

(52) U.S. Cl. 514/231.2; 514/248; 544/116; 544/235

(57) ABSTRACT

The present invention is directed to certain cinnoline compounds that are PDE10 inhibitors, pharmaceutical compositions containing such compounds and process for preparing such compounds. This invention is also directed to methods of treating diseases treatable by inhibition of PDE10 enzyme, such as obesity, non-insulin dependent diabetes, schizophrenia, bipolar disorder, obsessive-compulsive disorder, and the like.

### CROSS-REFERENCE

This application claims the benefit of U.S. Provisional Application No. 60/774,550, filed Feb. 21, 2006, the disclosure of which is incorporated herein by reference in its entirety.

### FIELD OF THE INVENTION

The present invention is directed to certain cinnoline compounds that are PDE10 inhibitors, pharmaceutical compositions containing such compounds and processes for preparing such compounds. This invention is also directed to methods of treating diseases treatable by inhibition of PDE10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE1 is stimulated by Ca²⁺/calmodulin. PDE2 activity is stimulated by cGMP. PDE3 is inhibited by cGMP. PDE4 is cAMP specific and is specifically inhibited by rolipram. PDE5 is cGMP-specific. PDE6 is expressed in retina.

PDE10 sequences were identified by using bioinformatics and sequence information from other PDE gene families (Fujishige et al., J. Biol. Chem. 274:18438-18445, 1999; Loughney et al., Gene 234:109-117, 1999; Soderling et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least three alternative exons encode N termini and two exons encode C-termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, for example, in neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, provided herein are compounds of Formula (I): 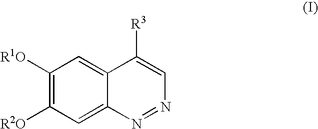
wherein:

R¹ and R² are independently selected from hydrogen, alkyl, or haloalkyl; and

R³ is a selected from formula (a)-(g): 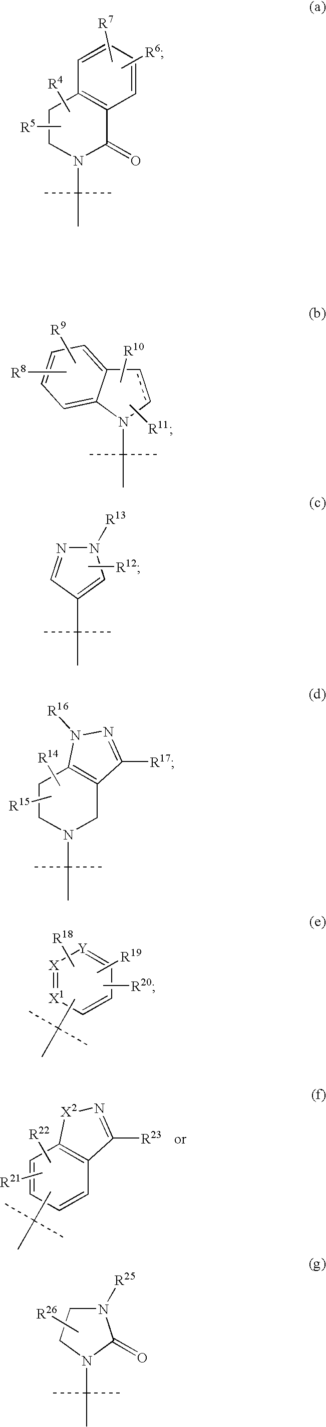
where:

X, X¹, and Y are all carbon; or one of X, X¹ and Y is carbon and the others are nitrogen; or two of X, X¹ and Y are carbon and the other is nitrogen;

X² is —NR²⁴—, —O—, or —S—;

dashed line in group (b) (shown as [symbol] is an optional double bond;

each R⁴, R⁵, R¹⁰, R¹¹, R¹⁴, and R¹⁵ is independently hydrogen or alkyl; or any R⁴ and R⁵, R¹⁰ and R¹¹, or R¹⁴ and R¹⁵, where feasible, form an oxo (═O) group;

each R¹⁸, R²¹, and R²² is independently hydrogen, alkyl, or halo; and

each R⁶, R⁷, R⁸, R⁹, R¹², R¹³, R¹⁶, R¹⁷, R¹⁹, R²⁰, R²³, R²⁴, R²⁵ and R²⁶ is independently hydrogen, alkyl, halo, haloalkyl, alkoxy, haloalkoxy, cyano, amino, monsubstituted or disubstituted amino, cycloalkylalkyl, aryl, heteroaryl, heterocyclyl, aralkyl, heteroaralkyl, heterocyclylalkyl, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸—R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl);

and wherein the aromatic or alicyclic ring in R⁶, R⁷, R⁸, R⁹, R¹², R¹³, R¹⁶, R¹⁷, R¹⁹, R²⁰, R²³, R²⁴, R²⁵, R²⁶, and R²⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, and optionally substituted heterocyclyl; and additionally substituted with one or two substitutents independently selected from R^{d} and R^{e} where R^{d} and R^{c} are hydrogen and fluoro; provided that R⁶, R⁷, R⁸, R⁹, R¹², R¹³, R¹⁶, R¹⁷, R¹⁹, R²⁰, R²³, R²⁴, R²⁵ and R²⁶ are not independently selected from hydrogen, alkyl, halo, cyano, haloalkyl, alkoxy, haloalkoxy, and amino.

In some embodiments, provided herein is a compound of Formula (I) as described above, or an individual stereoisomer, a mixture of stereoisomers, or a pharmaceutically acceptable salt thereof provided that the compound of Formula (I) is not:
7-(cyclopropylmethoxy)-2-(6,7-dimethoxycinnolin-4-yl)-6-methoxy-3,4-dihydro-isoquinolin-1(2H)-one;
6-(cyclopropylmethoxy)-7-(6,7-dimethoxycinnolin-4-yl)-6-methoxy-3,4-dihydro-isoquinolin-1(2H)-one;
1-(6,7-dimethoxycinnolin-4-yl)-N-ethylindoline-5-sulfonamide;
1-(6,7-dimethoxycinnolin-4-yl)-N,N-diethylindoline-5-sulfonamide;
1-(6,7-dimethoxycinnolin-4-yl)-N-(2-propyl)indoline-5-sulfonamide;
N-(cyclopropylmethyl)-1-(6,7-dimethoxycinnolin-4-yl)indoline-5-sulfonamide;
N-(methyl)-1-(6,7-dimethoxycinnolin-4-yl)indoline-5-sulfonamide;
6,7-dimethoxy-4-(5-(methylsulfonyl)indolin-1-yl)cinnoline;
4-(5-(furan-3-yl)indolin-1-yl)-6,7-dimethoxycinnoline;
1-(6,7-dimethoxycinnolin-4-yl)-N-methylindoline-5-sulfonamide;
1-(6,7-dimethoxycinnolin-4-yl)-N,N-dimethylindoline-5-sulfonamide;
4-(1-benzyl-1H-pyrazol-4-yl)-6,7-dimethoxycinnoline;
6,7-dimethoxy-4-(5-(thiophen-3-yl)-2,3-dihydro-1H-indolin-1-yl)cinnoline;
6,7-dimethoxy-4-(5-(pyrimidin-5-yl)indolin-1-yl)cinnoline;
1-(6,7-dimethoxycinnolin-4-yl)-N,N-diisopropylindoline-5-sulfonamide;
1-(6,7-dimethoxycinnolin-4-yl)-N-(2-morpholinoethyl)indoline-5-sulfonamide;
N-cyclopropyl-1-(6,7-dimethoxycinnolin-4-yl)indoline-5-sulfonamide;
6,7-dimethoxy-4-(5-(pyrrolidin-1-ylsulfonyl)-2,3-dihydro-1H-indolin-1-yl)cinnoline;
1-(6,7-dimethoxycinnolin-4-yl)-N-(2-methoxyethyl)indoline-5-sufonamide;
6,7-dimethoxy-4-(5-(pyridin-4-yl)indolin-1-yl)cinnoline;
4-(5-(3,5-dimethylisoxazol-4-yl)indolin-1-yl)-6,7-dimethoxycinnoline;
6,7-dimethoxy-4-(5-(piperidin-1-ylsulfonyl)indolin-1-yl)cinnoline;
3-(6,7-dimethoxycinnolin-4-yl)-N-ethylbenzamide;
N-cyclopropyl-3-(6,7-dimethoxycinnolin-4-yl)benzamide;
3-(6,7-dimethoxycinnolin-4-yl)-N,N-diethylbenzamide;
3-(6,7-dimethoxycinnolin-4-yl)-N-isobutylbenzamide;
6,7-dimethoxy-4-(5-(piperidin-1-ylcarbonyl)indolin-1-yl)cinnoline;
6-(benzyloxy)-2-(6,7-dimethoxycinnolin-4-yl)-3,4-dihydroisoquinolin-1(2H)-one;
N-cyclohexyl-3-(6,7-dimethoxycinnolin-4-yl)benzamide;
7-(cyclopropylmethoxy)-2-(6,7-dimethoxycinnolin-4-yl)-6-methoxy-3,4-dihydro-isoquinolin-1(2H)-one;
6-(cyclopropylmethoxy)-2-(6,7-dimethoxycinnolin-4-yl)-7-methoxy-3,4-dihydro-isoquinolin-1(2H)-one; and
2-(6,7-dimethoxycinnolin-4-yl)-5-(2-methoxyethoxy)-3,4-dihydroisoquinolin-1(2H)-one; or a pharmaceutically acceptable salt thereof.

In some embodiments where R³ is formula (b), the bond shown as [symbol] is a single bond.

In a second aspect, this invention is directed to a pharmaceutical composition comprising a compound of Formula (I) or a pharmaceutically acceptable salt thereof and a pharmaceutically acceptable expicient.

In a third aspect, this invention is directed to a method of treating a disorder treatable by inhibition of PDE10 enzyme in a patient which method comprises administering to the patient a pharmaceutical composition comprising a compound of Formula (I) or a pharmaceutically acceptable salt thereof and a pharmaceutically acceptable expicient. Preferably, the disease is obesity, non-insulin dependent diabetes, Huntington's disease, schizophrenia, bipolar disorder, or obsessive-compulsive disorder.

It will be readily apparent to a person skilled in the art that the pharmaceutical composition could contain one or more compounds of Formula (I) (including individual stereoisomers, mixtures of stereoisomers where the compound of Formula (I) has a stereochemical centre), a pharmaceutically acceptable salt thereof, or mixtures thereof.

### DETAILED DESCRIPTION

### Definitions

Unless otherwise stated, the following terms used in the specification and claims are defined for the purposes of this Application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring, e.g, cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated. Exemplary alkylenes include, e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfinyl” means a —SOR radical where R is alkyl as defined above, e.g., methylsulfinyl, ethylsulfinyl, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means an —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means a —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl as defined herein, and R′ is selected from hydrogen, alkyl, hydroxyalkyl, alkoxyalkyl, aryl, aralkyl, heteroaryl, heteroaralkyl, or haloalkyl, each as defined herein, e.g, aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means a —OR radical where R is aminoalkyl as defined above, e.g., 2-aminoethoxy, 2-dimethylaminopropoxy, and the like.

“Aminocarbonyl” means a —CONRR′ radical where R is independently hydrogen, alkyl, hydroxyalkyl, alkoxyalkyl, or aminoalkyl, each as defined herein, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined herein, e.g., —CONH₂, methylaminocarbonyl, 2-dimethylaminocarbonyl, and the like.

“Aminosulfonyl” means a —SO₂NRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —SO₂NH₂, methylaminosulfonyl, 2-dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, heterocyclylalkyl, each as defined herein, e.g., acetyl, propionyl, benzoyl, pyridinylcarbonyl, and the like.

“Acylamino” means a —NHCOR radical where R is alkyl, haloalkyl, cycloalkyl, cycloalkylalkyl, aryl, aralkyl, heteroaryl, heteroaralkyl, heterocyclyl, heterocyclylalkyl, each as defined herein, e.g., acetylamino, propionylamino, and the like.

“Aryl” means a monovalent monocyclic or bicyclic aromatic hydrocarbon radical of 6 to 12 ring atoms, e.g., phenyl, naphthyl or anthracenyl.

“Aralkyl” means an -(alkylene)-R radical where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantly, and the like.

“Cycloalkylalkyl” means an -(alkylene)-R radical where R is cycloalkyl as defined above; e.g., cyclopropylmethyl, cyclobutylmethyl, cyclopentylethyl, or cyclohexylmethyl, and the like.

“Cycloalkyloxy” means an —OR radical where R is cycloalkyl as defined, e.g., cyclopropyloxy, cyclobutyloxy, cyclopentyloxy, cyclohexyloxy, and the like.

“Cycloalkylalkyloxy” means an —OR radical where R is cycloalkylalkyl as defined, e.g., cyclopropylmethyloxy, cyclobutylmethyloxy, cyclopentylethyloxy, cyclohexylmethyloxy, and the like.

“Carboxy” means —COOH.

“Disubstituted amino” means a —NRR′ radical where R and R′ are independently alkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined herein, e.g., dimethylamino, phenylmethylamino, and the like.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

“Haloalkoxy” means an —OR radical where R is haloalkyl as defined above, e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means a —OR radical where R is hydroxyalkyl as defined above.

“Heterocyclyl” means a saturated or unsaturated monovalent monocyclic group of 3 to 8 ring atoms in which one or two ring atoms are heteroatom independently selected from N, O, and S(O)ₙ, where n is an integer from 0 to 2, the remaining ring atoms being C. Additionally, one or two ring carbon atoms can optionally be replaced by a —CO— group and the heterocyclic ring may be fused to phenyl or heteroaryl ring. Unless stated otherwise, the fused heterocyclyl ring can be attached at any ring atom. More specifically the term heterocyclyl includes, but is not limited to, pyrrolidino, piperidino, 2-oxopyrrolidinyl, 2-oxopiperidinyl, morpholino, piperazino, tetrahydropyranyl, thiomorpholino, and the like. When the heterocyclyl ring has five, six or seven ring atoms and is not fused to phenyl or heteroaryl ring, it is also referred to herein as “monocyclic five- six-, or seven membered heterocyclyl ring or five- six-, or seven membered heterocyclyl ring”. When the heterocyclyl ring is unsaturated it can contain one or two ring double bonds provided that the ring is not aromatic.

“Heterocyclylalkyl” means an -(alkylene)-R radical where R is heterocyclyl ring as defined above e.g., tetraydrofuranylmethyl, piperazinylmethyl, morpholinylethyl, and the like.

“Heteroaryl” means a monovalent monocyclic or bicyclic aromatic radical of 5 to 10 ring atoms where one or more, preferably one, two, or three, ring atoms are heteroatom independently selected from N, O, and S, the remaining ring atoms being carbon, e.g., benzofuranyl, thiophenyl, imidazolyl, oxazolyl, quinolineyl, furanyl, thiazolyl, pyridinyl, and the like.

“Heteroaralkyl” means an -(alkylene)-R radical where R is heteroaryl as defined above.

“Monosubstituted amino” means a —NHR radical where R is alkyl, acyl, sulfonyl, aryl, aralkyl, heteroaryl, heteroaralkyl, heterocyclyl, heterocyclylalkyl, hydroxyalkyl, alkoxyalkyl, or aminoalkyl, preferably alkyl, hydroxyalkyl, alkoxyalkyl, or aminoalkyl, each as defined herein, e.g., methylamino, 2-phenylamino, hydroxyethylamino, and the like.

The present invention also includes prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for instance, acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In certain embodiments, a “pharmaceutically acceptable salt” can include, for instance, salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are in general non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof, are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Oxo” means =(O) group.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with an alkyl group and situations where the heterocyclyl group is not substituted with the alkyl group.

“Optionally substituted phenyl” means a phenyl ring optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, and sulfinyl, each as defined herein.

“Optionally substituted heteroaryl” means a monovalent monocyclic or bicyclic aromatic radical of 5 to 10 ring atoms where one or more, preferably one, two, or three, ring atoms are heteroatoms independently selected from N, O, and S, the remaining ring atoms being carbon that is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, or carboxy, cycloalkyl, cycloalkylalkyl, cycloalkoxy, cycloalkylalkyloxy, and sulfinyl, each as defined herein. More specifically the term optionally substituted heteroaryl includes, but is not limited to, optionally substituted pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl.

“Optionally substituted heterocyclyl” means a saturated or unsaturated monovalent cyclic group of 3 to 8 ring atoms in which one or two ring atoms are heteroatoms independently selected from N, O, and S(O)ₙ, where n is an integer from 0 to 2, the remaining ring atoms being C. One or two ring carbon atoms can optionally be replaced by a —CO— group and is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, or carboxy, cycloalkyl, cycloalkylalkyl, cycloalkoxy, cycloalkylalkyloxy, and sulfinyl, each as defined herein.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical where R is alkyl, haloalkyl, aryl, aralkyl, heteroaryl, heteroaralkyl, heterocyclyl, heterocyclylalkyl, as defined above, e.g., methylsulfinyl, phenylsulfinyl, benzylsulfinyl, and the like.

“Sulfonyl” means a —SO₂R radical where R is alkyl, haloalkyl, aryl, aralkyl, heteroaryl, heteroaralkyl, heterocyclyl, heterocyclylalkyl, as defined above, e.g., methylsulfonyl, phenylsulfonyl, benzylsulfonyl, pyridinylsulfonyl, and the like.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

### EMBODIMENTS

In certain embodiments, a compound having Formula (I) as defined in the Summary of the Invention is provided.

(i). In one embodiment, provided herein is a compound of Formula (I) wherein R³ is a group of formula (a) as defined in the Summary of the Invention. Within this embodiment, one group of compounds is that wherein (a) is a group of formula: 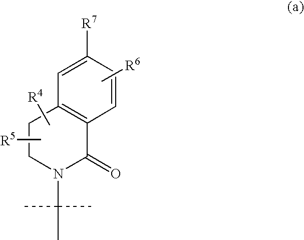
where R⁴, R⁵, R⁶ and R⁷ are as defined in the Summary of the Invention. Within this embodiment, one group of compounds is that wherein (a) is a group of formula: 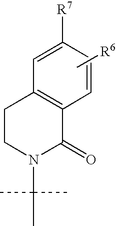
where one of R⁶ and R⁷ is hydrogen, alkyl, halo, haloalkyl, alkoxy, haloalkoxy, cyano, amino, monsubstituted or disubstituted amino, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸, R²⁹, R³⁰ and R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁶ and R⁷ is aryl, heteroaryl, or heterocyclyl; and wherein the aromatic or alicyclic ring in R⁶ and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Within this embodiment, one group of compounds is that wherein R⁷ is aryl, heteroaryl, or heterocyclyl optionally substituted with one to three substitutents independently selected from R^{a}, R^{b}, and R^{c}. Within this embodiment, one group of compounds is that wherein (a) is a group of 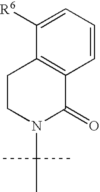
wherein R⁶ is aryl, heteroaryl, or heterocyclyl optionally substituted with one to three substitutents independently selected from R^{a}, R^{b}, and R^{c}.

(ii). In another embodiment, provided herein is a compound of Formula (I) wherein R³ is a group of formula (b) as defined in the Summary of the Invention. Within this embodiment, one group of compounds is that wherein (b) is a group of formula: 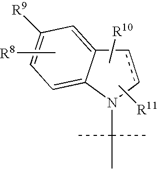
where R⁸, R⁹, R¹⁰ and R¹¹ are as defined in the Summary of the Invention. Within this embodiment, one group of compounds is that wherein (b) is a group of formula: 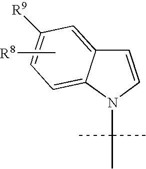
where one of R⁸ and R⁹ is hydrogen, alkyl, halo, haloalkyl, alkoxy, haloalkoxy, cyano, amino, monsubstituted or disubstituted amino, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸, R²⁹, R³⁰ and R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁸ and R⁹ is aryl, heteroaryl, or heterocyclyl; and wherein the aromatic or alicyclic ring in R⁸ and R⁹ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁹ is aryl, heteroaryl, or heterocyclyl optionally substituted with one to three substitutents independently selected from R^{a}, R^{b}, and R^{c}.

Within this embodiment, one group of compounds is that wherein (b) is a group of formula: 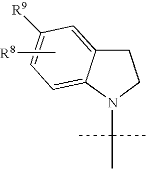
where R⁸ and R⁹ are as described immediately above provided that when R⁸ is hydrogen then R⁹ is not heteroaryl, alkylsulfonyl, —SO₂NR²⁷R³¹ where R³¹ is hydrogen or alkyl and R²⁷ is alkyl, alkoxyalkyl, unsubstituted cycloalkyl or cycloalkylalkyl, or unsubstituted heterocyclyl or heterocyclylalkyl. Within this embodiment, another group of compounds is that wherein (b) is a group of formula: 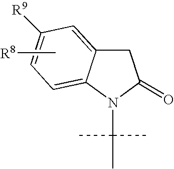
where R⁸ and R⁹ are as described immediately above.

(iii). In yet another embodiment, this invention is directed to a compound of Formula (I) wherein R³ is a group of formula (c) as defined in the Summary of the Invention. Within this embodiment, one class of compounds is that wherein (c) is a group of formula: 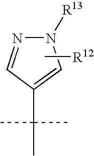
where R¹² is hydrogen or alkyl and R¹³ is aryl, heteroaryl, aralkyl, heteroaralkyl, or heterocyclyl optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. In one embodiment, R¹³ is aralkyl (preferably benzyl) optionally substituted with one to three substitutents independently selected from R^{a}, R^{b}, and R^{c}. In another embodiment, R¹³ is aralkyl (preferably benzyl) optionally substituted with one to three substitutents independently selected from R^{a}, R^{b}, and R^{c} provided that at least one of R^{a}, R^{b}, and R^{c} is other than hydrogen. In yet another embodiment, R¹³ is heteroaryl optionally substituted with one to three substitutents independently selected from R^{a}, R^{b}, and R^{c}. In one embodiment, R¹³ is heterocyclyl optionally substituted with optionally substituted phenyl, optionally substituted heteroaryl.

In one embodiment, (c) is a group of formula: 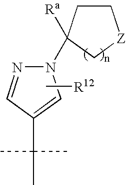
where R¹² is hydrogen or alkyl; n is 1, 2, or 3; Z is —O—, —NH— or —N-alkyl-; and R^{a} is optionally substituted phenyl or optionally substituted heteroaryl.

In one embodiment, (c) is a group of formula: 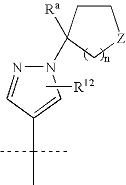
where R¹² is hydrogen; n is 1, 2, or 3; Z is —O—, —NH— or —N-alkyl-; and R^{a} is optionally substituted phenyl.

(iv). In yet another embodiment, this invention is directed to a compound of Formula (I) wherein R³ is a group of formula (d) as defined in the Summary of the Invention. Within this embodiment, one group of compounds is that wherein (d) is a group of formula: 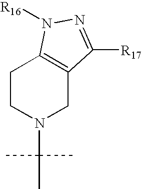
where one of R¹⁶ and R¹⁷ is hydrogen, alkyl, halo, haloalkyl, alkoxy, haloalkoxy, cyano, amino, monsubstituted or disubstituted amino, or —X³R²⁷ (where X³ is —O—, —CO—, —OC(O)—, —C(O)O, —NR²⁸CO—, —CONR²⁹, —S—, —SO—, —SO₂—, —NR³⁰SO₂—, or —SO₂NR³¹— where R²⁸-R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R¹⁶ and R¹⁷ is aryl, heteroaryl, or heterocyclyl; and wherein the aromatic or alicyclic ring in R¹⁶ and R¹⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R¹⁶ is aryl, heteroaryl, or heterocyclyl optionally substituted with one to three substitutents independently selected from R^{a}, R^{b}, and R^{c}.

(v). In yet another embodiment, this invention is directed to a compound of Formula (I) wherein R³ is a group of formula (e) as defined in the Summary of the Invention.

(A) Within this embodiment, one group of compounds is that wherein (e) is a group of formula: 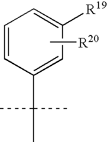
where one of R¹⁹ and R²⁰ is hydrogen, alkyl, halo, haloalkyl, alkoxy, haloalkoxy, cyano, amino, monsubstituted or disubstituted amino, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸, R²⁹, R³⁰ and R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R¹⁹ and R²⁰ is aryl, heteroaryl, or heterocyclyl; and wherein the aromatic or alicyclic ring in R¹⁹ and R²⁰ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl provided that when R²⁰ is hydrogen, then R¹⁹ is not —CONR²⁹R²⁷— where R²⁹ is hydrogen or alkyl, and R²⁷ is alkyl or unsubstituted cycloalkyl. Within this embodiment, in one group of compounds R¹⁹ is aryl, heteroaryl, or heterocyclyl optionally substituted with one to three substitutents independently selected from R^{a}, R^{b}, and R^{c}. Within this embodiment, in another group of compounds R¹⁹ is hydrogen and R²⁰ is mono or disubstituted amino and is located at the 4-position of the phenyl ring, the carbon atom of the phenyl ring attached to the cinnoline ring being the 1-position. Within this embodiment, in another group of compounds R¹⁹ is hydrogen, alkyl, or halo and R²⁰ is aryl, heteroaryl, or heterocyclyl optionally substituted with one to three substitutents independently selected from R^{a}, R^{b}, and R^{c} and is located at the 4-position of the phenyl ring, the carbon atom of the phenyl ring attached to the cinnoline ring being the 1-position.

(B) Within this embodiment, another group of compounds is that wherein (e) is a group of formula: 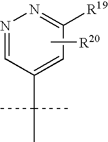
where R¹⁹ and R²⁰ are as defined in (A) above.

(C) Within this embodiment, another group of compounds is that wherein (e) is a group of formula: 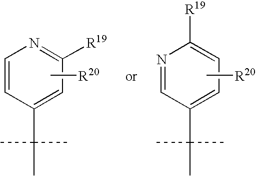
where R¹⁹ and R²⁰ are as defined in (A) above. Within this subgroup (C), one class of compounds is that where R¹⁹ is phenyl optionally substituted with one to three substitutents independently selected from R^{a}, R^{b}, and R^{c}. Within this subgroup (C), another class of compounds is that where R¹⁹ is heteroaryl optionally substituted with one to three substitutents independently selected from R^{a}, R^{b}, and R^{c}. Within this subgroup (C), another class of compounds is that where R¹⁹ is heterocyclyl, preferably piperazinyl, piperidinyl, or morpholinyl, optionally substituted with one to three substitutents independently selected from R^{a}, R^{b}, and R^{c}. Within the subgroups in this embodiment, in one class of compound R²⁰ is hydrogen, alkyl or halo.

(vi). In yet another embodiment, provided herein is a compound of Formula (I) wherein R³ is a group of formula (f) as defined in the Summary of the Invention. Within this embodiment, one group of compounds is that wherein (f) is a group of formula: 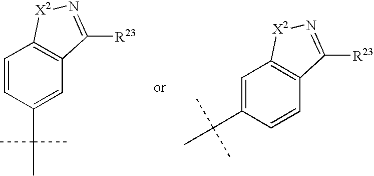
where X² is —O— or —NR²⁴—, preferably —NR²⁴— where one of R²³ and R²⁴ is hydrogen, alkyl, halo, haloalkyl, alkoxy, haloalkoxy, cyano, amino, monsubstituted or disubstituted amino, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸, R²⁹, R³⁰ and R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R²³ and R²⁴ is aryl, heteroaryl, or heterocyclyl; and wherein the aromatic or alicyclic ring in R²³ and R²⁴ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R²⁴ is aryl, heteroaryl, or heterocyclyl optionally substituted with one to three substitutents independently selected from R^{a}, R^{b}, and R^{c}. In another embodiment, X² is —S— and R²³ is alkyl, halo, haloalkyl, alkoxy, haloalkoxy, cyano, amino, monsubstituted or disubstituted amino, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸, R²⁹, R³⁰ and R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); wherein the aromatic or alicyclic ring in R²³ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

(vii). In yet another embodiment, provided herein is a compound of Formula (I) wherein R³ is a group of formula (g) as defined in the Summary of the Invention. Within this embodiment, one group of compounds is that wherein R²⁵ is hydrogen or alkyl and R²⁶ is aryl, heteroaryl, aralkyl, heteroaralkyl, or heterocyclyl optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. In one embodiment, R²⁶ is aralkyl (preferably benzyl) optionally substituted with one to three substitutents independently selected from R^{a}, R^{b}, and R^{c}. In another embodiment, R²⁶ is heteroaralkyl optionally substituted with one to three substitutents independently selected from R^{a}, R^{b}, and R^{c}.

(viii). In yet another embodiment, provided herein is a compound of Formula (I) wherein R³ is a group of formula: 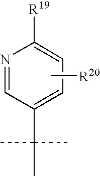
where R²⁰ is hydrogen, alkyl, or halo and R¹⁹ is mono- or disubstituted amino. Within this embodiment, one group of compounds is that where R²⁰ is alkyl or halo and R¹⁹ is monosubstitued amino. Within this embodiment, one group of compounds is that where R²⁰ alkyl or halo and R¹⁹ is disubstituted amino. Within the above groups, in one subgroup of compounds R²⁰ is at the C-3-position of the pyridine-5-yl ring.

In the above embodiments (i)-(viii), and subgroups/embodiments contained therein, one group of compounds is that where R¹ and R² are alkyl, preferably methyl.

In the above embodiments (i)-(viii), and subgroups/embodiments contained therein, another group of compounds is that where R¹ and R² are haloalkyl, preferably trifluoromethyl or difluoromethyl.

In the above embodiments (i)-(viii), and subgroups/embodiments contained therein, another group of compounds is that where R¹ is haloalkyl and R² is alkyl, preferably R¹ is trifluoromethyl, difluoromethyl, or 2,2,2-trifluoroethyl and R² is methyl.

(ix). In yet another embodiment, this invention is directed to a compound of Formula (I) where R¹ and R² are independently alkyl, haloalkyl or hydrogen; preferably alkyl or haloalkyl; and R³ is a group of formula (a)-(f) where R⁴, R⁵, R¹⁰, R¹¹, R¹², R¹⁴, R¹⁵, R¹⁸, R²¹, and R²² are hydrogen or R¹⁰ and R¹¹, or R¹⁴ and R¹⁵ form an oxo (═O) group; and one of R⁶, R⁷, R⁸, R⁹, R¹⁶, R¹⁷, R¹⁹, R²⁰, R²³, and R²⁴ is hydrogen, alkyl, or halo and the other of R⁶, R⁷, R⁸, R⁹, R¹⁶, R¹⁷, R¹⁹, R²⁰, R²³, and R²⁴ and R¹³is monsubstituted or disubstituted amino, aryl, heteroaryl, heterocyclyl, aralkyl, or —X³R²⁷ (where X³ is —O—, —C(O)O, —CONR²9-, or —SO₂— where R²⁷ is alkyl, cycloalkyl, cycloalkylalkyl, aryl, aralkyl, or heteroaralkyl, and R²⁹ is hydrogen or alkyk); and wherein the aromatic or alicyclic ring in R⁶, R⁷, R⁸, R⁹, R¹⁶, R¹⁷, R¹⁹, R²⁰, R²³, and R²⁴ and R¹³ and R²⁷ is optionally substituted with one to three substitutents independently selected from R^{a}, R^{b}, and R^{c} which are alkyl, alkoxy, halo, haloalkyl, haloalkoxy, cyano, carboxy, hydroxyl, alkoxycarbonyl, monosubstituted amino, disubstituted amino, optionally substituted heteroaryl, or optionally substituted phenyl.

Within this embodiment, one group of compounds is that wherein R³ is 3-morpholin-4-ylphenyl; 4-piperidin-1-ylphenyl; 3-ethylsulfonylphenyl; 6-(piperidin-1-yl)-3,4-dihydroisoquinolin-1(2H)-one; 3-(1-methyl-1H-pyrazol-4-yl)phenyl; 1-phenyl-1H-pyrazol-4-yl; 3-(cyclopropylaminocarbonyl)-4,5,6,7-tetrahydro-1H-pyrazolo[4,3-c]pyridin-5-yl; 3-(4,4-dimethyl-4,5-dihydrol,3-oxazol-2-yl)phenyl; 3-(4,4-dimethyl-4,5-dihydro-1,3-oxazol-2-yl)pyridin-5-yl; 1-(3-methoxyphenyl)-1H-pyrazol-4-yl; 1-(3-ethoxyphenyl)-1H-pyrazol-4-yl; 3-(ethoxycarbonyl)-1H-indazol-5-yl; 3-(ethoxycarbonyl)-1H-indazol-6-yl; 3-acetylaminophenyl; 3-dimethylaminophenyl; 3-(thien-3-yl)phenyl; 3-(furan-3-yl)phenyl; 3-(4,4-dimethyl-4,5-dihydro-1,3-thiazol-2-yl)phenyl; 3-(cyclopropylaminocarbonyl)-1H-indazol-6-yl; 5-(morpholin-4-yl)indol-1-yl; 1-(4-methylbenzyl)-1H-pyrazol-4-yl; 1-(4-tert-butylbenzyl)-1H-pyrazol-4-yl; 1-(4-phenylbenzyl)-1H-pyrazol-4-yl; 1-(4-methoxycarbonylbenzyl)-1H-pyrazol-4-yl; 1-(2-phenylbenzyl)-1H-pyrazol-4-yl; 1-(3-trifluoromethylbenzyl)-1H-pyrazol-4-yl; 1-(2-trifluoromethylbenzyl)-1H-pyrazol-4-yl; 1-(2-cyanobenzyl)-1H-pyrazol-4-yl; 1-(3-methylbenzyl)-1H-pyrazol-4-yl; 1-(4-cyanobenzyl)-1H-pyrazol-4-yl; 1-(2-methylbenzyl)-1H-pyrazol-4-yl; 1-(4-trifluoromethoxybenzyl)pyrazol-4-yl; 6-(morpholin-4-yl)pyridin-3-yl; 2-(4-methylpiperazin-1-yl)pyridin-4-yl; 1-(4-fluorobenzyl)-1H-pyrazol-4-yl; 3-(dimethylaminocarbonyl)-1H-indazol-6-yl; 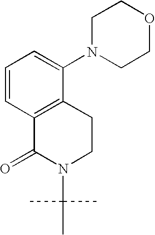
5-morpholino-3,4-dihydroisoquinolin-(2H)-one ;
3-cyclopropylaminocarbonylbenzo[d]isothiazol-5-yl; 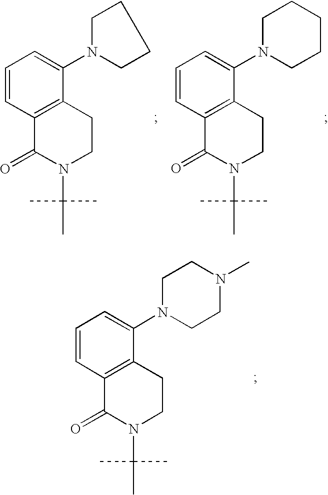
3-fluoro-2-morpholin-4-ylpyridin-4-yl; 2-(1-methylpiperazin-4-yl)pyrimidin-5-yl; 1-(2-fluorobenzyl)-1H-pyrazol-4-yl; 2-(piperidin-1-yl)pyridin-5-yl; 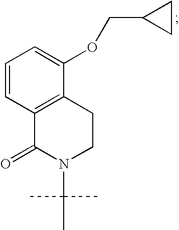
2-(1-methylpiperazin-4-yl)pyridin-5-yl; 3-cyclopropylaminocarbonyl-benzo[d]isothiazol-6-yl; 3-cyclopropylaminocarbonylbenzo[d]isothiazol-7-yl; 2-(piperazin-1-yl)pyridin-4-yl; 2-(piperazin-1-yl)pyridin-5-yl; 3-ethoxycarbonylbenzo-[d]isoxazol-5-yl; 3-ethoxycarbonylbenzo[d]isoxazol-6-yl; 2-(2-oxo-1-methylpiperazin-4-yl)pyridin-4-yl; 2-(3-methoxypyrrolidin-1-yl)pyridin-5-yl; 2-(4,4-difluoropiperidin-1-yl)pyridin-5-yl; 2-(3S-methylmorpholin-4-yl)pyridin-5-yl; 2-(3-methoxypiperidin-1-yl)pyridin-4-yl; 2-(isopropylamino)pyridin-5-yl; 2-(2-methylpropylamino)pyridin-5-yl; 2-[—NHCH(CH₃)CH₂CH₃]pyridin-5-yl; 2-(2-methoxyethylamino)pyridin-5-yl; 2-(2-aminoethylamino)pyridin-5-yl; 2-(pyrrolidin-1-yl)pyridin-5-yl; 2-(1-tert-butoxycarbonylazetidin-4-ylmethylamino)pyridin-5-yl; 2-(ethyl-n-propylamino)pyridin-5-yl; 2-(2R,6S-dimethylmorpholin-4-yl)pyridin-5-yl; 4-(quinolin-2-ylmethyloxy)phenyl; 2-(4-methoxypiperidin-1-yl)pyridin-5-yl; 2-(2-oxo-1-methylpiperazin-1-yl)pyridin-5-yl; 2-(4-fluoropiperidin-1-yl)pyridin-5-yl; 2-(3-(trifluoromethyl)-5,6,7,8-tetrahydro-[1,2,4]triazolo[4,3-a]pyrazin-7-yl)pyridin-5-yl; 2-(methylisopropylamino)pyridin-5-yl; 2-(4-methoxyazetidin-1-yl)pyridin-5-yl; 3-benzyloxyphenyl; 2-(4-oxopiperidin-1-yl)pyridin-5-yl; 2-[—N(CH₃)CH₂CH(CH₃)₂]pyridin-5-yl; 2-(4,4-difluoroazetidin-1-yl)pyridin-5-yl; 2-(4-methylaminopiperidin-1-yl)pyridin-5-yl; 3-diethylaminocarbonylbenzo[d]isothiazol-6-yl; 2-(phenylamino)pyridin-5-yl; 2-(oxazol-2-yl)phenyl; 3-isopropylaminocarbonylbenzo[d]isothiazol-6-yl; 2-(2-tert-butoxyethylamino)pyridin-5-yl; 2-(S—NHCH(CH₃)CH₂OCH₃)pyridin-5-yl; 2-[(methyl)-(2-tert-butylethylamino)amino]pyridin-5-yl; 2-(phenoxy)pyridin-5-yl; 2-(4-dimethylaminophenylamino)pyridin-5-yl; 2-(cyclopropylmethyloxy)pyridin-5-yl; 2-(1-tert-butoxycarbonyl-1,2,5,6-tetrahydropyridin-4-yl)pyridin-5-yl; 2-(2-ethoxyethylamino)pyridin-5-yl; 2-[(isopropyl)-(2-tert-butylaminoethyl)aminopyridin-5-yl; 2-(2-pyridin-2-ylethylamino)pyridin-5-yl; 2-(dimethylamino)pyridin-5-yl; 2-(3-cyanophenylamino)pyridin-5-yl; 2-(1,2,5,6-tetrahydropyridin-4-yl)pyridin-5-yl; 2-(2-bromo-4,5,6,7-tetrahydrothiazolo[5,4-c]pyridin-1-yl)pyridin-5-yl; 2-(3-fluorobenzylamino)pyridin-5-yl; 2-(thiophen-2-ylmethylamino)pyridin-5-yl; 3-fluoro-4-acetylphenyl; 4-(4-carboxyazetidin-1-yl)phenyl; 2-(pyridin-3-ylmethylamino)pyridin-5-yl; 2-(3-tert-butylphenylamino)pyridin-5-yl; 2-(1-methylbenzylamino)pyridin-5-yl; 2-(2,2-dimethylpropylamino)pyridin-5-yl; 2-(1-methyl-1,2,5,6-tetrahydropyridin-4-yl)pyridin-5-yl; 2-(n-butylamino)pyridin-5-yl; 2-(4-hydroxy-4-phenylpiperidin-1-yl)pyridin-5-yl; 2-(4-carboxyazetidin-1-yl)pyridin-5-yl; 2-(3R,5S-dimethylpiperazin-4-yl)pyridin-5-yl; 2-[methyl-(2-pyridin-2-ylethyl)amino]pyridin-5-yl; 2-[methyl-(2-phenylethyl)amino]pyridin-5-yl; 2-(2-methylbenzo[d]thiazol-6-ylamino)pyridin-5yl; 2-(2-hydroxy-2-methylpropylamino)pyridin-5-yl; 2-(4-cyano-4-phenylpiperidin-1-yl)pyridin-5-yl; 2-(2-amino-2-methylpropylamino)pyridin-5-yl; 2-(4-(oxazol-5-yl)phenylamino)pyridin-5-yl; 2-[5-(cyclopropyl)-[1.3.4]-thiadiazol-2-ylamino]pyridin-5-yl; 2-(2-indol-3-ylethylamino)pyridin-5-yl; 2-(benzothiophen-2-ylmethylamino)pyridin-5-yl; 2-(3-trifluoromethoxybenzylamino)pyridin-5-yl; 6-isopropylamino-2-methylpyridin-3-yl; 2-(2-pyridin-4-ylethylamino)pyridin-5-yl; 2-(2-pyridin-3-ylethylamino)pyridin-5-yl; 2-(1S-methylbenzylamino)pyridin-5-yl; 2-(1R-methylbenzylamino)pyridin-5-yl; 2-(1RS-methylbenzylamino)pyridin-5-yl; 2-(2-phenylethylamino)pyridin-5-yl; 2-[2-(2-methoxyphenylethyl)amino]pyridin-5-yl; 2-(5-methylfuran-2-ylmethylamino)pyridin-5-yl; 2-(pyridin-2-ylmethylamino)pyridin-5-yl; 2-[2-(3-methoxyphenyl)ethylamino]-pyridin-5-yl; 2-(2-phenylpropylamino)pyridin-5-yl; 3-(morpholin-4-yl)phenyl; 4-(piperidinyl-1-yl)phenyl; 2-[N-(2-tert-butylaminoethyl)-N-methylamino]pyridin-5-yl; 2-(4-dimethylaminophenylamino)pyridin-5-yl; 2-[N-(2-tert-butylaminoethyl)-N-(2-propyl)amino]pyridin-5-yl; 2-(4-oxazol-5-ylphenylamino)pyridin-5-yl; 2-(5-cyclopropyl-[1.3.4]-thiazol-2-ylamino)pyridin-5-yl; 2-(3-trifluoromethoxybenzylamino)pyridin-5-yl; 2-[1-(4-fluorophenyl)propylamino)pyridin-5-yl; 2-[2-(3-methoxyphenyl)ethylamino]-pyridin-5-yl; 2-(2-phenylpropylamino)pyridin-5-yl; 4-fluoro-3-methylcarbonylaminophenyl; 4-(4-hydroxypiperidin-1-yl)pyridin-5-yl; 3-methyl-2-(2-isopropylamino)pyridin-5-yl; 2-(pyridin-4-ylmethylamino)pyridin-5-yl; 2-[1-(pyridin-2-)ylmethyl)ethylamino)pyridin-5-yl; 2-[1-(tert-butyloxycarbonyl)pyrrolidin-3S-yl]pyridin-5-yl; 3-fluoro-2-(isopropylamino)pyridin-5-yl; or 3-fluoro-2-[3-fluoro-2-(isopropylamino)pyridin-5-yl]pyridin-5-yl.

Representative compounds of Formula (I) are provided in Table 1 below:

TABLE 1

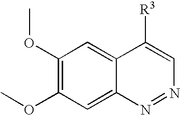

| Cpd | |
|---|---|
| # | R³ |
| 1 | 3-morpholin-4-ylphenyl |
| 2 | 4-piperidin-1-ylphenyl |
| 3 | 3-ethylsulfonylphenyl |
| 4 | 6-(piperidin-1-yl)-3,4-dihydroisoquinolin-1(2H)-one |
| 5 | 3-(1-methyl-1H-pyrazol-4-yl)phenyl•HCO₂H |
| 6 | 1-phenyl-1H-pyrazol-4-yl•HCO₂H |
| 7 | 3-(cyclopropylaminocarbonyl)-4,5,6,7-tetrahydro-1H-pyrazolo |
| | [4,3-c]-pyridin-5-yl |
| 8 | 3-(4,4-dimethyl-4,5-dihydro 1,3-oxazol-2-yl)phenyl |
| 9 | 3-(4,4-dimethyl-4,5-dihydro-1,3-oxazol-2-yl)pyridin-5-yl |
| 10 | 1-(3-methoxyphenyl)-1H-pyrazol-4-yl•HCO₂H |
| 11 | 1-(3-ethoxyphenyl)-1H-pyrazol-4-yl•HCO₂H |
| 12 | 3-(ethoxycarbonyl)-1H-indazol-5-yl |
| 13 | 3-(ethoxycarbonyl)-1H-indazol-6-yl |
| 14 | 3-acetylaminophenyl•HCO₂H |
| 15 | 3-dimethylaminophenyl•HCO₂H |
| 16 | 3-(thien-3-yl)phenyl•HCO₂H |
| 17 | 3-(furan-3-yl)phenyl•HCO₂H |
| 18 | 3-(4,4-dimethyl-4,5-dihydro-1,3-thiazol-2-yl)phenyl |
| 19 | 3-(cyclopropylaminocarbonyl)-1H-indazol-6-yl |
| 20 | 5-(morpholin-4-yl)indol-1-yl•HCO₂H |
| 21 | 1-(4-methylbenzyl)-1H-pyrazol-4-yl |
| 22 | 1-(4-tert-butylbenzyl)-1H-pyrazol-4-yl |
| 23 | 1-(4-phenylbenzyl)-1H-pyrazol-4-yl |
| 24 | 1-(4-methoxycarbonylbenzyl)-1H-pyrazol-4-yl |
| 25 | 1-(2-phenylbenzyl)-1H-pyrazol-4-yl |
| 26 | 1-(3-trifluoromethylbenzyl)-1H-pyrazol-4-yl |
| 27 | 1-(2-trifluoromethylbenzyl)-1H-pyrazol-4-yl |
| 28 | 1-(2-cyanobenzyl)-1H-pyrazol-4-yl |
| 29 | 1-(3-methylbenzyl)-1H-pyrazol-4-yl |
| 30 | 1-(4-cyanobenzyl)-1H-pyrazol-4-yl |
| 31 | 1-(2-methylbenzyl)-1H-pyrazol-4-yl |
| 32 | 1-(4-trifluoromethoxybenzyl)pyrazol-4-yl |
| 33 | 6-(morpholin-4-yl)pyridin-3-yl |
| 34 | 2-(4-methylpiperazin-1-yl)pyridin-4-yl |
| 35 | 1-(4-fluorobenzyl)-1H-pyrazol-4-yl•HCO₂H |
| 36 | 3-fluoro-2-(isopropylamino)pyridin-5-yl |
| 37 | 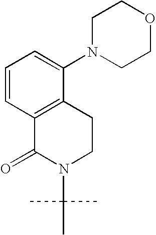 |
| | 5-morpholino-3,4-dihydroisoquinolin-(2H)-one |
| 38 | 3-cyclopropylaminocarbonylbenzo[d]isothiazol-5-yl |
| 39 | 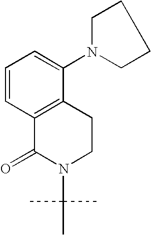 |
| 40 | 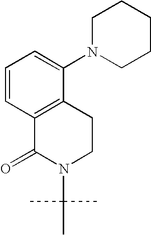 |
| 41 | 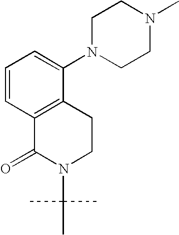 |
| 42 | 3-fluoro-2-morpholin-4-ylpyridin-4-yl |
| 43 | 2-(1-methylpiperazin-4-yl)pyrimidin-5-yl |
| 44 | 1-(2-fluorobenzyl)-1H-pyrazol-4-yl•HCO₂H |
| 45 | 2-(piperidin-1-yl)pyridin-5-yl |
| 46 | 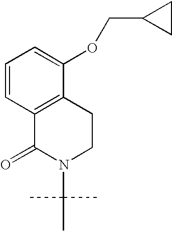 |
| 47 | 2-(1-methylpiperazin-4-yl)pyridin-5-yl |
| 48 | 3-cyclopropylaminocarbonylbenzo[d]isothiazol-6-yl |
| 49 | 3-cyclopropylaminocarbonylbenzo[d]isothiazol-7-yl |
| 50 | 2-(piperazin-1-yl)pyridin-4-yl |
| 51 | 2-(piperazin-1-yl)pyridin-5-yl |
| 52 | 3-ethoxycarbonylbenzo[d]isoxazol-5-yl |
| 53 | 3-ethoxycarbonylbenzo[d]isoxazol-6-yl |
| 54 | 2-(2-oxo-1-methylpiperazin-4-yl)pyridin-4-yl |
| 55 | 2-(3-methoxypyrrolidin-1-yl)pyridin-5-yl |
| 56 | 2-(4,4-difluoropiperidin-1-yl)pyridin-5-yl |
| 57 | 2-(3S-methylmorpholin-4-yl)pyridin-5-yl |
| 58 | 2-(3-methoxypiperidin-1-yl)pyridin-4-yl |
| 59 | 2-(isopropylamino)pyridin-5-yl |
| 60 | 2-(2-methylpropylamino)pyridin-5-yl |
| 61 | 2-[—NHCH(CH₃)CH₂CH₃]pyridin-5-yl |
| 62 | 2-(2-methoxyethylamino)pyridin-5-yl |
| 63 | 2-(2-aminoethylamino)pyridin-5-yl |
| 64 | 2-(pyrrolidin-1-yl)pyridin-5-yl |
| 65 | 2-(1-tert-butoxycarbonylazetidin-4-ylmethylamino)pyridin-5-yl |
| 66 | 2-(ethyl-n-propylamino)pyridin-5-yl |
| 67 | 2-(2R,6S-dimethylmorpholin-4-yl)pyridin-5-yl |
| 68 | 4-(quinolin-2-ylmethyloxy)phenyl |
| 69 | 2-(4-methoxypiperidin-1-yl)pyridin-5-yl |
| 70 | 2-(2-oxo-1-methylpiperazin-1-yl)pyridin-5-yl |
| 71 | 2-(4-fluoropiperidin-1-yl)pyridin-5-yl |
| 72 | 2-(3-(trifluoromethyl)-5,6,7,8-tetrahydro-[1,2,4]triazolo[4,3-a] |
| | pyrazin-7-yl)pyridin-5-yl |
| 73 | 2-(methylisopropylamino)pyridin-5-yl |
| 74 | 2-(4-methoxyazetidin-1-yl)pyridin-5-yl |
| 75 | 3-benzyloxyphenyl |
| 76 | 2-(4-oxopiperidin-1-yl)pyridin-5-yl |
| 77 | 2-[—N(CH₃)(CH₂CH(CH₃)₂)]pyridin-5-yl |
| 78 | 2-(4,4-difluoroazetidin-1-yl)pyridin-5-yl |
| 79 | 2-(4-methylaminopiperidin-1-yl)pyridin-5-yl |
| 80 | 3-diethylaminocarbonylbenzo[d]isothiazol-6-yl |
| 81 | 2-(phenylamino)pyridin-5-yl |
| 82 | 2-(oxazol-2-yl)phenyl |
| 83 | 3-isopropylaminocarbonylbenzo[d]isothiazol-6-yl |
| 84 | 2-(2-tert-butoxyethylamino)pyridin-5-yl |
| 85 | 2-(S—NHCH(CH₃)CH₂OCH₃)pyridin-5-yl |
| 86 | 2-[(methyl)-(2-tert-butylethylamino)amino]pyridin-5-yl |
| 87 | 2-(phenoxy)pyridin-5-yl |
| 88 | 2-(4-dimethylaminophenylamino)pyridin-5-yl |
| 89 | 2-(cyclopropylmethyloxy)pyridin-5-yl |
| 90 | 2-(1-tert-butoxycarbonyl-1,2,5,6-tetrahydropyridin-4-yl)pyridin-5- |
| | yl |
| 91 | 2-(2-ethoxyethylamino)pyridin-5-yl |
| 92 | 2-[(isopropyl)-(2-tert-butylaminoethyl)aminopyridin-5-yl |
| 93 | 2-(2-pyridin-2-ylethylamino)pyridin-5-yl |
| 94 | 2-(dimethylamino)pyridin-5-yl |
| 95 | 2-(3-cyanophenylamino)pyridin-5-yl |
| 96 | 2-(1,2,5,6-tetrahydropyridin-4-yl)pyridin-5-yl |
| 97 | 2-(2-bromo-4,5,6,7-tetrahydrothiazolo[5,4-c]pyridin-1-yl)pyridin-5- |
| | yl |
| 98 | 2-(3-fluorobenzylamino)pyridin-5-yl•TFA |
| 99 | 2-(thiophen-2-ylmethylamino)pyridin-5-yl |
| 100 | 3-fluoro-4-acetylphenyl |
| 101 | 4-(4-carboxyazetidin-1-yl)phenyl |
| 102 | 2-(pyridin-3-ylmethylamino)pyridin-5-yl |
| 103 | 2-(3-tert-butylphenylamino)pyridin-5-yl |
| 104 | 2-(1-methylbenzylamino)pyridin-5-yl |
| 105 | 2-(2,2-dimethylpropylamino)pyridin-5-yl |
| 106 | 2-(1-methyl-1,2,5,6-tetrahydropyridin-4-yl)pyridin-5-yl |
| 107 | 2-(n-butylamino)pyridin-5-yl |
| 108 | 2-(4-hydroxy-4-phenylpiperidin-1-yl)pyridin-5-yl |
| 109 | 2-(4-carboxyazetidin-1-yl)pyridin-5-yl |
| 110 | 2-(3R,5S-dimethylpiperazin-1-yl)pyridin-5-yl |
| 111 | 2-[methyl-(2-pyridin-2-ylethyl)amino]pyridin-5-yl |
| 112 | 2-[methyl-(2-phenylethyl)amino]pyridin-5-yl |
| 113 | 2-(2-methylbenzo[d]thiazol-6-ylamino)pyridin-5yl |
| 114 | 2-(2-hydroxy-2-methylpropylamino)pyridin-5-yl |
| 115 | 3-fluoro-2-[3-fluoro-2-(isopropylamino)pyridin-5-yl]pyridin-5-yl |
| 116 | 2-(4-cyano-4-phenylpiperidin-1-yl)pyridin-5-yl |
| 117 | 2-(2-amino-2-methylpropylamino)pyridin-5-yl |
| 118 | 2-(4-(oxazol-5-yl)phenylamino)pyridin-5-yl |
| 119 | 2-[5-(cyclopropyl)-[1.3.4]-thiadiazol-2-ylamino]pyridin-5-yl |
| 120 | 2-(2-indol-3-ylethylamino)pyridin-5-yl•TFA |
| 121 | 2-(benzothiophen-2-ylmethylamino)pyridin-5-yl•TFA |
| 122 | 2-(3-trifluoromethoxybenzylamino)pyridin-5-yl•TFA |
| 123 | 6-isopropylamino-2-methylpyridin-3-yl |
| 124 | 2-(2-pyridin-4-ylethylamino)pyridin-5-yl•2TFA |
| 125 | 2-(2-pyridin-3-ylethylamino)pyridin-5-yl•2TFA |
| 126 | 2-(1S-methylbenzylamino)pyridin-5-yl•TFA |
| 127 | 2-(1R-methylbenzylamino)pyridin-5-yl•TFA |
| 128 | 2-(1RS-methylbenzylamino)pyridin-5-yl |
| 129 | 2-(2-phenylethylamino)pyridin-5-yl |
| 130 | 2-[2-(2-methoxyphenylethyl)amino]pyridin-5-yl•TFA |
| 131 | 2-(5-methylfuran-2-ylmethylamino)pyridin-5-yl |
| 132 | 2-(pyridin-2-ylmethylamino)pyridin-5-yl•2TFA |
| 133 | 2-[2-(3-methoxyphenyl)ethylamino]pyridin-5-yl•TFA |
| 134 | 2-(2-phenylpropylamino)pyridin-5-yl•TFA |
| 135 | 3-(morpholin-4-yl)phenyl |
| 136 | 4-(piperidinyl-1-yl)phenyl |
| 137 | 2-[N-(2-tert-butylaminoethyl)-N-methylamino]pyridin-5-yl |
| 138 | 2-(4-dimethylaminophenylamino)pyridin-5-yl |
| 139 | 2-[N-(2-tert-butylaminoethyl)-N-(2-propyl)amino]pyridin-5-yl |
| 140 | 2-(4-oxazol-5-ylphenylamino)pyridin-5-yl |
| 141 | 2-(5-cyclopropyl-[1.3.4]-thiazol-2-ylamino)pyridin-5-yl |
| 142 | 2-(3-trifluoromethoxybenzylamino)pyridin-5-yl•TFA |
| 143 | 2-[1-(4-fluorophenyl)ethylamino)pyridin-5-yl•TFA |
| 144 | 2-[2-(3-methoxyphenyl)ethylamino]pyridin-5-yl•TFA |
| 145 | 2-(2-phenylpropylamino)pyridin-5-yl•TFA |
| 146 | 4-fluoro-3-methylcarbonylaminophenyl |
| 147 | 2-[1-(tert-butyloxycarbonyl)pyrrolidin-3S-yl]pyridin-5-yl |
| 148 | 4-(4-hydroxypiperidin-1-yl)pyridin-5-yl |
| 149 | 3-methyl-2-(2-isopropylamino)pyridin-5-yl |
| 150 | 2-(pyridin-4-ylmethylamino)pyridin-5-yl |
| 151 | 2-[1-(pyridin-2-ylmethyl)ethylamino)pyridin-5-yl•TFA |

General Synthetic Schemes

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons,.1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (I) where R¹, R² and R³ are as defined in the Summary of the Invention can be prepared as described in Scheme 1 below.

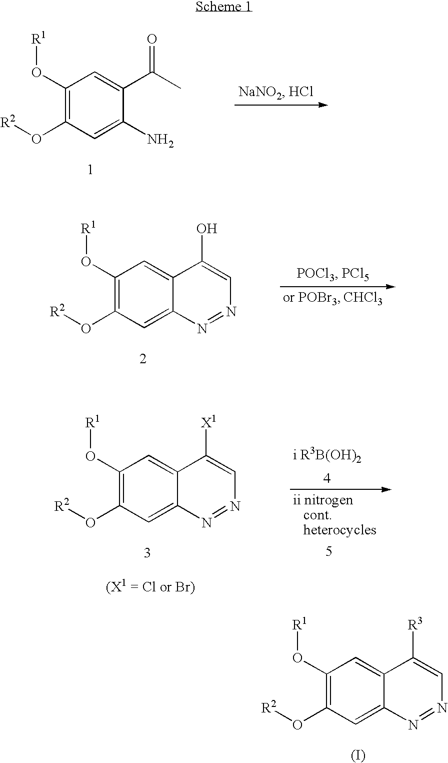

Treatment of 2-amino-4,5-dialkoxyacetophenones 1 with sodium nitrite in concentrated HCl and water provides diazo compound intermediates that cyclize upon heating to provide 6,7-dialkoxy-4-hydroxycinnolines 2. Treatment of 2 with either phosphorous oxychloride or phosphorous oxybromide provides the corresponding chloro or bromo compound of formula 3.

The chloro derivative is prepared by heating 2 in neat phosphorous oxychloride, followed by recrystallization of the product after neutralization (see Castle et al., J. Org. Chem. 17:1571, 1952). The bromo derivative is prepared by mixing a concentrated suspension of the 4-hydroxycinnoline in chloroform and phosphorous oxybromide at room temperature and then warming to reflux for 8 to 16 h. Extractive workup after neutralization and subsequent recrystallization from alcoholic solvent such as ethanol provides 4-bromocinnoline.

Compounds of formula 1 are either commercially available (e.g., 2-amino-4,5-dimethoxyacetophenone) or can be synthesized by methods well known in the art. For example, simple dialkyl ethers, wherein the alkyl groups at the 3,4-positions are the same, can be readily prepared under standard etherification reaction conditions. For example, 3,4-dihydroxyacetophenone can be treated with an excess of a base such as cesium carbonate and the desired alkyl halide to directly provide the dialkylated product. Other bases such as triethylamine, sodium hydride, potassium carbonate, potassium hydride, etc. can be employed in combination with a variety of solvents such as acetone, acetonitrile, DMF, and THF, and the like. 2-Amino-4,5-dialkoxyacetophenones 1 is prepared by nitration with nitric acid in one of several solvents including acetic acid or sulfuric acid at ice bath temperatures to provide 2-nitro-4,5-dialkoxyacetophenones (Iwamura et al., Bioorg. Med. Chem. 10:675, 2002). Reduction of the nitro group under known reaction conditions e.g., hydrogenation with palladium on carbon, iron powder in acetic acid, or nickel boride, among others, provides the desired compound 1. (Castle et al., J. Org. Chem. 19:1117, 1954).

Compounds of formula 1 where R¹ and R² are different can also be prepared by methods well known in the art. For example if the desired substituent at the 3-position is the methyl ether, acetovanillone (3-methoxy-4-hydroxyacetophenone) can be utilized as a starting material. Simple etherification, as described above, can be utilized to provide the required 4-substitution, followed by nitration and reduction steps as described above. Alternatively, compounds of formula 1 can be prepared under Mitsunobu reaction conditions by treating phenol with diethyl or diisopropyl azo-dicarboxylates, triphenylphosphine, and the desired alkyl alcohol in THF solution to give the corresponding alkoxy derivative. Treatment of the phenol with haloacetic acid e.g., chlorodifluoroacetic acid under basic conditions provides difluoromethyl ether.

If compounds of formula 1 where R¹ is other than methyl is desired, 3,4-dihydroxyacetophenone can be utilized as the starting material. 3,4-Dihydroxyacetophenone can be selectively protected as its 4-benzyl ether (Greenspan et al., J. Med. Chem. 42:164, 1999) by treatment with benzyl bromide and lithium carbonate in DMF solution. Functionalization of the 3-OH group with the desired alkyl halide can be accomplished under the esterification conditions described above, including Mitsunobu reaction. Removal of the benzyl ether by hydrogenolysis with palladium on carbon in alcoholic solvents such as methanol and followed by etherification of the 4-OH yields the 3,4-dialkoxyacetophenones. Nitration of 3,4-dialkoxyacetophenones, followed by reduction of the nitro group provides the desired compound 1.

4-Bromo-6,7-bis-difluoromethoxycinnoline analogs can be prepared from 3,4-dimethoxyacetophenone by reaction with nitric acid to yield 3,4-dimethoxy-6-nitroacetophenone which upon treatment with pyridine-HCl provides 1-(4,5-dihydroxy-2-nitrophenyl)ethanone. Treatment of 1-(4,5-dihydroxy-2-nitrophenyl)ethanone with chlorodifluoroacetic acid provides 1-(4,5-bis(difluoromethoxy)-2-nitrophenyl)ethanone which upon reduction of the nitro group to amino group followed by cyclization under conditions described above provides the desired compound.

Compound 3 is then converted to a compound of Formula (I) where R³is a group of formula (a)-(c) by reacting it with aryl or heteroaryl boronic acids under Suzuki coupling reaction conditions.

Compounds of Formula (I) where R³ is a group of formula (a), (b) where the dashed line is not a bond, or (d), can be prepared by reacting 3 where X¹ is halo or other suitable leaving group such as tosylate, triflate, mesylate and the like with the corresponding heterocyclic ring in the presence of a base such as triethylamine, pyridine, and the like. Suitable solvents include, and the not limited to, tetrahydrofuran, DMF, and the like. Alternatively, such compounds can be prepared by heating 3 with the heterocyclic ring in a suitable organic solvent such as THF, benzene, dioxane, toluene, alcohol, or mixtures thereof, under catalytic conditions using, for example, a palladium or copper catalyst (such as, but not limited to tris(dibenzylideneacetone)-dipalladium(o) or copper (I) iodide) in the presence of a suitable base such as potassium carbonate, sodium t-butoxide, lithium hexamethyldisilizane, and the like.

Compounds of formulae 4 and 5 are either commercially available or they can be prepared by methods well known in the art. For example, 3-(morpholino)phenyl boronic acid pinacol ester and 1-[4-(4,4,5,5-tetramethyl-1,3,2-dioxaborolan-2-yl) are available from Maybridge Chemicals, (Cornwall, UK). 3-Ethylsulfonylphenyl boronic acid and 4-[5-(4,4,5,5-tetramethyl-[1,3,2]dioxoborolan-2-yl)pyridin-2-yl]morpholine may be purchased from Frontier Scientific (Logan, UT) and 3-(N,N-dimethylamino)phenyl boronic acid is available from Acros (Geel, Belgium).

Other boronic acids, such as those utilized to prepare the compounds set forth in Scheme 2 below, may readily be prepared from the corresponding bromides as follows. Bromobenzoic acid can be converted to the corresponding oxazoline, thiazoline, or imidazoline substituted derivative by treatment of the corresponding acid chloride with the appropriate amino alcohol, as shown in scheme below. Subsequent eaction with butyl lithium and B(O-iPr)₃ provides the desired boronic acid derivative.

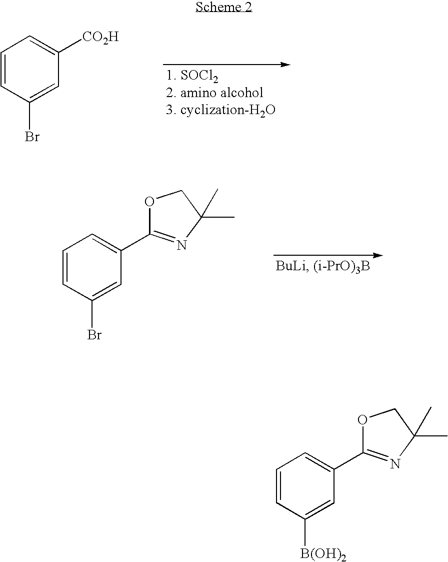

Indazole boronic acids can be prepared in the same manner, starting from the corresponding bromo-indazoles, which are commercially available from J&W PharmaLab (Morrisville, Pa.). Alternatively, the boronic esters can be produced by treatment of the aryl bromides under palladium catalysis with bis-pinacol borane or the like.

The amino substituted 3,4-dihydroisoquinoline-1(2H)-ones can readily be accessed via the corresponding bromides, which are commercially available from J&W PharmaLab (Morrisville, Pa.) by palladium catalyzed Buchwald/Hartwig couplings with the desired secondary amines. The benzyl bromides utilized in Example 10 can be readily obtained from a number of commercial sources, include Aldrich Chemical Co. (Milwaukee, Wis.)

Utility and Methods of Use

In one aspect, methods are provided for treating a disorder or disease treatable by inhibition of PDE10 comprising administering a therapeutically effective amount of compound as provided herein to a patient in need thereof to treat the disorder or disease.

The compounds of the present invention inhibit PDE10 enzyme activity and hence raise the levels of cAMP or cGMP within cells that express PDE10. Accordingly, inhibition of PDE10 enzyme activity can be useful in the treatment of diseases caused by deficient amounts of cAMP or cGMP in cells. PDE10inhibitors can also be of benefit in cases wherein raising the amount of cAMP or cGMP above normal levels results in a therapeutic effect. Inhibitors of PDE10 can be used to treat disorders of the peripheral and central nervous system, cardiovascular diseases, cancer, gastro-enterological diseases, endocrinological diseases and urological diseases.

Indications that can be treated with PDE10 inhibitors, either alone or in combination with other drugs, include, but are not limited to, those diseases thought to be mediated in part by the basal ganglia, prefrontal cortex and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention can be useful in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways. (Saxena S. et al., Br. J. Psychiatry Suppl., 1998; (35):26-37.) Neurons in these pathways project to striatal neurons that express PDE10. PDE10inhibitors cause cAMP to be elevated in these neurons; elevations in cAMP result in an increase in CREB phosphorylation and thereby improve the functional state of these neurons. The compounds of the present invention can therefore be useful for the indication of OCD. OCD may result, in some cases, from streptococcal infections that cause autoimmune reactions in the basal ganglia (Giedd JN et al., Am J Psychiatry., 2000 February; 157(2):281-3). Because PDE10inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that since PDE10degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention can be useful for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention provides methods for dealing with memory loss separate from dementia, including mild cognitive impairment (MCI) and age-related cognitive decline. The present invention provides methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention can be useful in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1(ataxin-1); spinocerebellar ataxia type-2 (ataxin-2); spinocerebellar ataxia type-3 also called Machado-Joseph disease, MJD (ataxin- 3); spinocerebellar ataxia type-6 (alpha 1a-voltage dependent calcium channel); spinocerebellar ataxia type-7 (ataxin-7); and spinal and bulbar muscular atrophy, SBMA, also know as Kennedy disease (androgen receptor).

The basal ganglia are important for regulating the function of motor neurons; disorders of the basal ganglia result in movement disorders. Most prominent among the movement disorders related to basal ganglia function is Parkinson's disease (Obeso et al., Neurology., 62(1 Suppl 1):S17-30, 2004). Other movement disorders related to dysfunction of the basla ganglia include tardive dyskinesia, progressive supranuclear palsy and cerebral palsy, corticobasal degeneration, multiple system atrophy, Wilson disease, and dystonia, tics, and chorea. The compounds of the invention can be used to treat movement disorders related to dysfunction of basal ganglia neurons.

PDE10 inhibitors can be used to raise cAMP or cGMP levels and prevent neurons from undergoing apoptosis. PDE10inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity will inhibit cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention may be used to stop the growth of cancer cells that express PDE10.

The compounds of the invention are also suitable for use in the treatment of diabetes and related disorders such as obesity, by focusing on regulation of the cAMP signaling system. By inhibiting PDE-10A activity, intracellular levels of cAMP and increased, thereby increasing the release of insulin-containing secretory granules and, therefore, increasing insulin secretion. See, for example, WO 2005/012485, which is hereby incorporated by reference in its entirety. The compounds of Formula (I) can also be used to treat the diseases disclosed in U.S. Patent application publication No. 2006/019975, the disclosure of which is incorporated herein by reference in its entirety.

Testing

The PDE10 inhibitory activities of the compounds of the present invention can be tested, for example, using the in vitro and in vivo assays described in working Biological Examples below.

Administration and Pharmaceutical Compositions

In general, the compounds of this invention can be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. Therapeutically effective amounts of compounds of formula (I) may range from approximately 0.1-1000 mg per day; preferably 0.5 to 250 mg/day, more preferably 3.5 mg to 70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

The compounds can be administered as the sole active agent or in combination with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic ml and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range and can be administered either simultaneously or sequentially.

Drugs suitable in combination with the compounds of the present invention include, but not limited to, other suitable schizophrenia drugs such as Clozaril, Zyprexa, Risperidone, and Seroquel, bipolar disorder drugs such as Lithium, Zyprexa, and Depakote, Parkinson's disease drugs such as Levodopa, Parlodel, Permax, Mirapex, Tasmar, Contan, Kemadin, Artane, and Cogentin, agents used in the treatment of Alzheimer's disease such as, but not limited to, Reminyl, Cognex, Aricept, Exelon, Akatinol, Neotropin, Eldepryl, Estrogen and Cliquinol, agents used in the treatment of dementia such as, but not limited to, Thioridazine, Haloperidol, Risperidone, Cognex, Aricept, and Exelon, agents used in the treatment of epilepsy such as, but not limited to, Dilantin, Luminol, Tegretol, Depakote, Depakene, Zarontin, Neurontin, Barbita, Solfeton, and Felbatol, agents used in the treatment of multiple sclerosis such as, but not limited to, Detrol, Ditropan XL, OxyContin, Betaseron, Avonex, Azothioprine, Methotrexate, and Copaxone, agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g., agonists, antagonists, such as Rosiglitazone, Troglitazone and Pioglitazone), insulin secretagogues (for example, sulfonylurea drugs (such as Glyburide, Glimepiride, Chlorpropamide, Tolbutamide, and Glipizide) and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors and 11beta-HSD inhibitors), hepatic glucose output lowering compounds (such as glucagon antagonists and metaformin, such as Glucophage and Glucophage XR), insulin and insulin derivatives (both long and short acting forms and formulations of insulin), and anti-obesity drugs (such as β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat)).

### EXAMPLES

The following preparations and examples are given to enable those skilled in the art to more clearly understand and to practice the present invention. They should not be considered as limiting the scope of the invention, but merely as being illustrative and representative thereof. All spectra were recorded at 300 MHz on a Bruker Instruments NMR unless otherwise stated. Coupling constants (J) are in Hertz (Hz) and peaks are listed relative to TMS (δ 0.00 ppm). Microwave reactions were performed using a Personal Chemistry Optimizer™ microwave reactor in 10 mL Personal Chemistry microwave reactor vials. All reactions were performed at 200° C. for 600 s with the fixed hold time ON unless otherwise stated. Sulfonic acid ion exchange resins (SCX) were purchased from Varian Technologies. Analytical HPLC was performed on 4.6 mm×100 mm Waters Sunfire RP C 18 5 μm column using (i) a gradient of 20/80 to 80/20 acetonitrile (0.1% formic acid)/water (0.1% formic acid) over 6 min (Method A), (ii) a gradient of 20/80 to 80/20 acetonitrile (0.1% formic acid)/water (0.1% formic acid) over 8 min (Method B), (iii) a gradient of 40/60 to 80/20 acetonitrile (0.1% formic acid)/water (0.1% formic acid) over 6 min (Method C), or (iv) a gradient of 40/60 to 80/420 acetonitrile (0.1% formic acid)/water (0.1% formic acid) over 8 min (Method D). Preparative HPLC was performed on 30 mm×100 mm Xtera Prep RP₁₈5μ columns using an 8 min gradient of 95/5 to 20/80 water (0.1% formic acid)/acetonitrile (0.1% formic acid).

### Synthetic Examples

### Example 1

### Synthesis of 4-bromo-6,7-dimethoxycinnoline

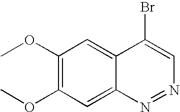 Step 1

1-(2-Amino-4,5-dimethoxyphenyl)ethanone (15.60 g, 0.07991 mol) was dissolved in concentrated hydrogen chloride in water (555 mL) and water (78 mL). The reaction mixture was cooled to −5° C. (ice/brine) and a solution of sodium nitrite (5.55 g, 0.0804 mol) in water (20 mL) was added over a period of 45 min. The reaction mixture was stirred another 1 h at 0° C. and then warmed to 60-75° C. for 4 h. The reaction mixture was then cooled to room temperature using an ice bath and the resulting precipitate was collected via filtration. The solid hydrochloride salt thus obtained was added to approximately 1.0 L of water and then basified to pH ˜12 with sodium hydroxide. The resulting brown solution was neutralized with hydrochloric acid, and the resulting precipitate was collected to provide 12.77 g of 6,7-dimethoxycinnolin-4-ol as a light tan solid (78% yield), which was used without further purification.

Step 2

To a solution of 6,7-dimethoxycinnolin-4-ol (2.00 g, 0.00970 mol), prepared as described above in Step 1, in chloroform (20 mL) was added phosphorus oxybromide (12.2 g, 0.0426 mol). Brief solvation was observed for 10 min after addition of the phosphorus oxybromide and then a suspension formed. The reaction mixture was stirred for 8 h at room temperature, and was then heated to reflux for 18 h. The reaction mixture was poured onto crushed ice (resulting in gas evolution), warmed to room temperature (giving a volume of around 125 mL) and neutralized to ˜pH 7 with saturated sodium acetate. The mixture was then extracted with dichloromethane and the combined organics were dried (MgSO₄), filtered, and concentrated. Recrystallization from absolute ethanol provided 1.30 g of 4-bromo-6,7-dimethoxycinnoline as light yellow superfine fibrous crystals. MS [M+]=269, [M+2]=271, ¹H NMR (DMSO d₆), δ (ppm), 9.38 (s, 1H), 7.77 (s, 1H), 7.21 (s, 1H), 4.03 (s, 6H).

### Example 2

### Synthesis of 6,7-dimethoxy-4-(3-morpholin-4-ylphenyl)cinnoline

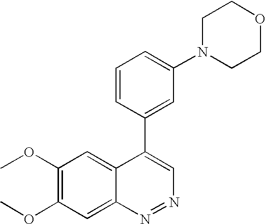

Into a 5 mL microwave tube was added 4-bromo-6,7-dimethoxycinnoline (50.1 mg, 0.186 mmol, prepared as described above in Example 1), 3-(morpholino)phenylboronic acid pinacol ester (60.1 mg, 0.208 mmol), bis(triphenylphosphine)palladium(II) chloride (27.3 mg, 0.0389 mmol), aqueous sodium carbonate (2.00 M, 140 μL) and a mixture of dimethoxyethane: water:ethanol (900 μL, 7:3:2). The resulting suspension was subjected to microwave radiation at 140° C. for 5.0 minutes. The reaction product was filtered through Celite, which was rinsed with ethyl acetate (20 mL). The combined organic layers were washed with aqueous saturated sodium bicarbonate (15 mL) and brine, dried (sodium sulfate), filtered, and volatiles were removed in vacuo. The residue was dissolved in methanol (3 mL) and loaded onto an SCX column (0.34 g). The SCX column was rinsed several times with two column volumes of methanol and the product was eluted using 7.0 M ammonia in methanol (5 mL). Volatiles were removed in vacuo to afford 19.1 mg (29.2%) of 6,7-dimethoxy-4-(3-morpholin-4-ylphenyl)cinnoline as a light yellow solid. ¹H NMR (CDCl₃), d 9.07 (s, 1H), 7.80 (s, 1H), 7.47 (t, J=7.5 Hz, 1H), 7.18 (s, 1H), 7.07 (m, 3H), 4.13 (s, 3H), 3.93 (s, 3H), 3.90 (t, J=7.5 Hz, 4H), 3.25 (t, J=7.5 Hz, 4H), LC/MS (EI), t_{R} 5.5 min (Method B), m/z 352.2 (M⁺+1).

The following compounds were prepared in a similar manner to Example 2 using different starting materials:

### 6,7-Dimethoxy-4-(4-piperidin-1-ylphenyl)cinnoline

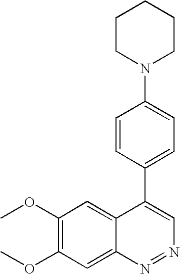

Prepared using 1-[4-(4,4,5,5-tetramethyl-1,3,2-dioxaborolan-2-yl)phenyl]piperidine. The product was initially purified by HPLC column chromatography (using a 20-80% gradient of acetonitrile:water (with 0.1% formic acid) at a flow rate of 45 mL/min) before the SCX column chromatography. 21.4 mg (32.5% yield). LC/MS (EI), t_{R} 5.5 min (Method B), m/z 350.2 (M⁺+1).

### 4-[3-(Ethylsulfonyl)phenyl]-6,7-dimethoxycinnoline

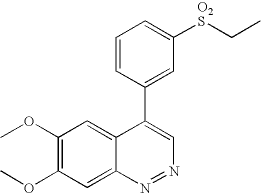

Prepared using 3-ethylsulfonylphenyl boronic acid to give 65 mg of above compound. LC/MS (EI), t_{R} 3.12 min (Method D), m/z 359 (M⁺+1).

### 6,7-Dimethoxy-4-(6-morpholin-4-ylpyridin-3-yl)cinnoline

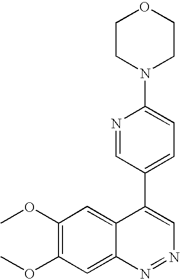

Prepared using 4-[5-(4,4,5,5-tetramethyl-[1,3,2]dioxoborolan-2-yl)pyridin-2-yl]morpholine. The product was also purified by rotary chromatography (elution with chloroform to 10% methanol in chloroform gradient) to give 63 mg of above compound. LC/MS (EI), t_{R} 2.51 min (Method D), m/z 353 (M⁺+1).

### 6,7-Dimethoxy-4-[2-(4-methylpiperazin-1-yl)pyridin-4-yl]cinnoline

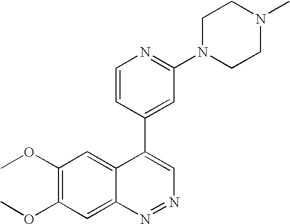

Prepared using 1-methyl-4-[4-(4,4,5,5-tetramethyl-[1,3,2]dioxoborolan-2-yl)pyridine-2-yl]morpholine. The product was also purified by rotary chromatography (elution with chloroform to 10% methanol in chloroform gradient) to give 60 mg of above compound. LC/MS (EI), t_{R} 1.81 min (Method D), m/z 366 (M⁺+1).

### 4-[3-(4,4-Dimethyl-4,5-dihydro-1,3-oxazol-2-yl)phenyl]-6,7-dimethoxycinnoline

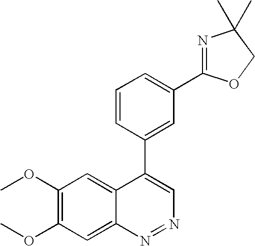

Prepared using [3-(4,4-dimethyl-4,5-dihydro-1,3-oxazol-2-yl)phenyl]boronic acid. The product was also purified by rotary chromatography (elution with chloroform to 10% methanol in chloroform gradient) to give 56 mg of above compound. LC/MS (EI), t_{R} 3.18 min (Method D), m/z 364 (M⁺+1).

### 4-[5-(4,4-Dimethyl-4,5-dihydro-1,3-oxazol-2-yl)pyridin-3-yl]-6,7-dimethoxycinnoline

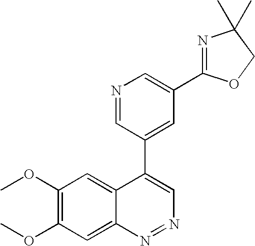

Prepared using [5-(4,4-dimethyl-4,5-dihydro-1,3-oxazol-2-yl)pyridine-3-yl]boronic acid. The product was also purified by rotary chromatography (elution with chloroform to 10% methanol in chloroform gradient) to give 53 mg of above compound. LC/MS (EI), t_{R} 3.12 min (Method D), m/z 365 (M⁺+1).

### Example 3

### Synthesis of N-[3-(6,7-dimethoxycinnolin-4-yl)phenyl]acetamide hydroformate

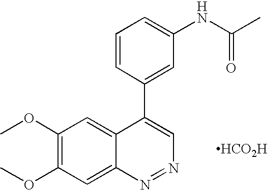

Into a microwave tube was added 4-bromo-6,7-dimethoxycinnoline (200 mg, 0.0008 mol, prepared as described in Example 1 above), [3-(acetylamino)phenyl]boronic acid (100 mg, 0.0008 mol), bis(triphenylphosphine) palladium(II) chloride (95.6 mg, 0.136 mmol), aqueous sodium carbonate (2.00 M, 0.28 mL) and a mixture of dimethoxyethane:water:ethanol (5 mL, 7:3:2). The resulting suspension was subjected to microwave radiation at 140° C. for 600 seconds. The reaction was filtered through celite, which was washed with methanol. Concentration, followed by ISCO chromatographic purification (using a gradient of 50% ethyl acetate:hexanes to 100% ethyl acetate) afforded 190 mg of N-[3-(6,7-dimethoxycinnolin-4-yl)phenyl]acetamide hydroformate as a yellow solid. ¹H NMR (CDCl₃), d 9.16 (s, 0.5H), 9.06 (s, 1H), 7.91-7.88 (s, 1H), 7.80 (s, 1H), 7.66 (s, 0.5H), 7.60-7.49 (m, 2H), 7.35-7.29 (m, 2H), 4.16 (s, 1H), 4.12 (s, 3H), 3.98 (s, 3H), 3.77 (s, 3H), LC/MS (EI), t_{R} 4.76 min (Method A), m/z 324 (M⁺+1).

The following compounds were prepared in a similar manner to Example 3 using different starting materials:

### 3-(6,7-Dimethoxycinnolin-4-yl)-N,N-dimethylaniline hydroformate

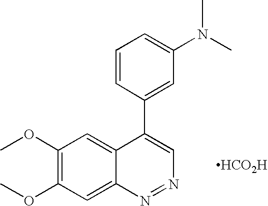

Prepared using 3-(N,N-dimethylamino)phenyl boronic acid to give 190 mg of above compound. LC/MS (EI), t_{R} 5.93 min (Method A), m/z 310 (M⁺+1).

### 4-[3-(4,4-Dimethyl-4,5-dihydro-1,3-thiazol-2-yl)phenyl]-6,7-dimethoxycinnoline

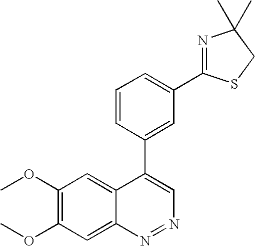

Prepared using 3-(4,4-dimethyl-4,5-dihydro-1,3-thiazol-2-yl)phenyl]boronic acid to give 26 mg of above compound. LC/MS (EI), t_{R} 4.39 min (Method D), m/z 380 (M⁺+1).

### Ethyl 5-(6,7-dimethoxycinnolin-4-yl)-1H-indazole-3-carboxylate

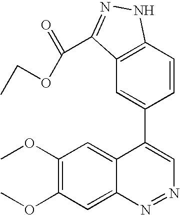

Prepared using ethyl 5-(4,4,5,5-tetramethyl-1,3,2-dioxaborolan-2-yl)-1H-indazole-3-carboxylate. Column chromatography (using an eluent of 1:1 ethylacetate/hexane (3 column volumes) followed by a 3-5% gradient methanol/dichloromethane) gave 83 mg of above compound. LC/MS (EI), t_{R} 5.27 min (Method B), m/z 379.0 (M⁺+1).

### Ethyl 6-(6,7-dimethoxycinnolin-4-yl)-1H-indazole-3-carboxylate

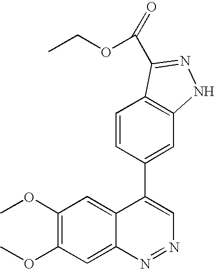

Prepared using ethyl 6-(4,4,5,5-tetramethyl-1,3,2-dioxaborolan-2-yl)-1H-indazole-3-carboxylate. Column chromatography (using an eluent of 1:1 ethylacetate/hexane (3 column volumes) followed by a 3-5% gradient of methanol/dichloromethane) gave 144 mg (of above compound. LC/MS (EI), t_{R} 5.75 min (Method B), m/z 379 (M⁺+1).

### Example 4

### Synthesis of 6,7-dimethoxy-4-[3-(1-methyl-1H-pyrazol-4-yl)phenyl]cinnoline hydroformate

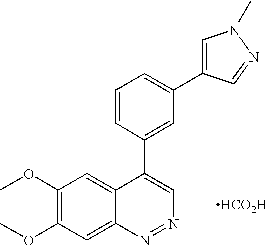 Step 1

4-bromo-6,7-dimethoxycinnoline (200 mg, 0.8 mmol, prepared as described in Example 1 above), bis(triphenylphosphine)palladium(II) chloride (95.6 mg, 0.136 mmol), aqueous sodium carbonate (2.00 M, 0.28 mL), 3-bromophenyl boronic acid (200 mg, 0.8 mol) and a mixture of 1,2-dimethoxyethane:water:ethanol (5 mL, 7:3:2) were added to a microwave tube and sealed. The resulting suspension was subjected to microwave radiation at 140° C. for 10 minutes. The reaction contents were filtered through celite, which was washed with methanol and dichloromethane and the organics were concentrated. Purification by ISCO chromatography (using 50% ethyl acetate:hexane, followed by 100% ethyl acetate) afforded 190 mg of 4-(3-bromophenyl)-6,7-dimethoxyxinnoline as a yellow solid.

Step 2

4-(3-Bromophenyl)-6,7-dimethoxycinnoline (50 mg, 0.1 mmol, prepared as described in Step 1 above), bis(triphenylphosphine)palladium(II) chloride (17.8 mg, 0.0253 mmol), 1-methyl-4-(4,4,5,5-tetramethyl-1,3,2-dioxaborolan-1H-pyrazole (30 mg, 0.1 mmol), 2.00 M of sodium carbonate in water (0.052 mL) and a mixture of 1,2-dimethoxyethane:water:ethanol (0.9 mL, 7:3:2) were added to a microwave tube and sealed and irradiated in a microwave reactor. The reaction contents were filtered through celite, which was washed with methanol and dichloromethane and the organics were concentrated. The residue was dissolved in methanol (1 mL). Purification by preparative HPLC (using a gradient of 20-80% acetonitrile with 0.1% formic acid) afforded 9 mg of 6,7-dimethoxy-4-[3-(1-methyl-1H-pyrazol-4-yl)phenyl]cinnoline hydroformate as a yellow solid. ¹H NMR (CDCl₃), d 9.10 (s, 1H), 8.10 (s, 0.5H), 7.83 (d, J=5.46 Hz, 2H), 7.69-7.55 (m, 4H), 7.41 (d, J=7.39 Hz, 1H), 7.17 (s, 1H), 4.14 (s, 3H), 3.98 (s, 3H), 3.92 (s, 3H), LC/MS (EI), t_{R} 5.3 min (Method A), m/z 347 (M⁺+1).

The following compounds were prepared in a similar manner to Example 4 using different starting materials:

### 4-[3-(3-Furanyl)phenyl]-6,7-dimethoxycinnoline hydroformate

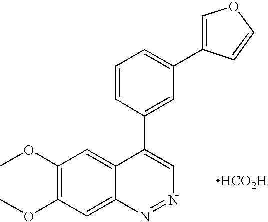

Prepared using furan-3-boronic acid to give 1.3 mg of above compound. LC/MS (EI), t_{R} 7.71 min (Method A), m/z 343 (M⁺+1).

### 6,7-Dimethoxy-4-[3-(3-thienyl)phenyl]cinnoline hydroformate

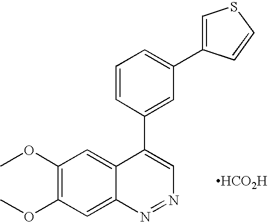

Prepared using 3-thienyl boronic acid to give 1.2 mg of above compound. LC/MS (EI), t_{R} 7.7 min (Method A), m/z 349 (M⁺+1).

### Example 5

### Synthesis of 6,7-dimethoxy-4-(1-phenyl-1H-pyrazol-4-yl)cinnoline hydroformate

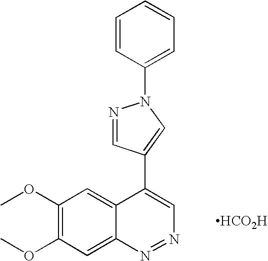

A mixture of 6,7-dimethoxy-4-(1H-pyrazol-4-yl)cinnoline (50 mg, 0.2 mmol, prepared as described in Example 10, Step 1), phenylboronic acid (35.7 mg, 0.293 mmol), cupric acetate (35.5 mg, 0.196 mmol), triethylamine (0.134 mL, 0.965 mmol), pyridine (0.128 mL) and 1,4-dioxane (1.55 mL) was stirred at room temperature for 40 h. Water (15 mL) and ethyl acetate (25 mL) were added, and the mixture was filtered through celite. The organic layer was separated, washed with brine (15 mL), dried (sodium sulfate), and concentrated in vacuo. The residue was purified by preparative HPLC (using a gradient of 20-80% acetonitrile with 0.1% formic acid) to afford 5 mg (8% yield) of 6,7-dimethoxy-4-(1-phenyl-1H-pyrazol-4-yl)cinnoline hydroformate as a brown solid. ¹H NMR (CDCl₃), d 9.16 (s, 1H), 8.32 (s, 1H), 8.14 (s, 1H), 7.82 (par obs s, 2H), 7.79 (par obs s, 1H), 7.58-7.53 (m, 2H), 7.43-7.38 (m, 2H), 4.14 (s, 3H), 4.04 (s, 3H), LC/MS (EI), t_{R} 6.09 min (Method A), m/z 333 (M⁺+1).

The following compounds were prepared in a similar manner to Example 5 using different starting materials:

### 6,7-Dimethoxy-4-[1-(3-methoxyphenyl)-1H-pyrazol-4-yl]cinnoline hydroformate

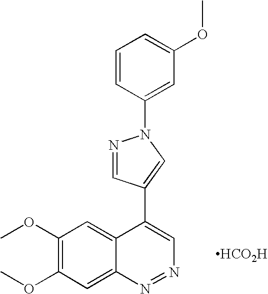

Prepared using 3-methoxyphenyl boronic acid to give 1 mg of above compound. LC/MS (EI) t_{R} 6.2 min (Method A), m/z 363 (M⁺+1).

### 4-[1-(3-Ethoxyphenyl)-1H-pyrazol-4-yl]-6,7-dimethoxycinnoline hydroformate

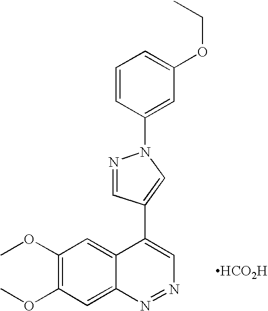

Prepared using 3-ethoxyphenyl boronic acid to give 1.8 mg of above compound. LC/MS (EI), t_{R} 6.76 min (Method A), m/z 377 (M⁺+1).

### Example 6

### 2-(6,7-dimethoxycinnolin-4-yl)-6-piperidin-1-yl-3,4-dihydroisoquinolin-1(2H)-one

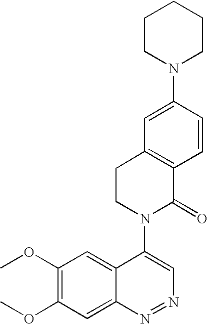

4-Bromo-6,7-dimethoxycinnoline (127.7 mg, 0.4746 mmol, prepared as described in Example 1 above), 6-piperidin-1-yl-3,4-dihydroisoquinolin-1(2H)-one (130.8 mg, 0.5679 mmol), copper(I) iodide (8.4 mg, 0.044 mmol), potassium carbonate (132.0 mg, 0.9551 mmol), N,N′-dimethyl-1,2-ethanediamine (20 μL) and toluene (0.6 mL) were added to a 5 mL microwave tube, and the resulting suspension was heated at 115° C. for 23 h. The reaction was filtered thru celite, which was washed with ethyl acetate (20 mL). The compound was purified by preparative HPLC column chromatography (using a gradient of 35-80% acetonitrile:water (with 0.1% formic acid) and a flow rate of 45 mL/min). SCX column chromatography (using 7.0 M of ammonia in methanol (8 mL) as eluent) afforded 71.8 mg of 2-(6,7-dimethoxycinnolin-4-yl)-6-piperidin-1-yl-3,4-dihydroisoquinolin-1(2H)-one a yellow solid, which contained 4.9 wt % dichloromethane by ¹H NMR. ¹H NMR (CDCl₃), d 9.10 (s, 1H), 8.10 (s, 0.5H), 7.83 (d, J=5.46 Hz, 2H), 7.69-7.55 (m, 4H), 7.41 (d, J=7.39 Hz, 1H), 7.17 (s, 1H), 4.14 (s, 3H), 3.98 (s, 3H), 3.92 (s, 3H), LC/MS (EI), t_{R} 5.2 min (Method D), m/z 419.2 (M⁺+1).

### Example 7

### Synthesis of N-cyclopropyl-6-(6,7-dimethoxycinnolin-4-yl)-1H-indazole-3-carboxamide

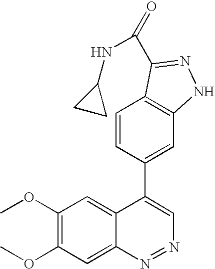 Step 1

Into a 10 ml microwave tube was added 4-bromo-6,7-dimethoxycinnoline (150 mg, 0.56 mmol, prepared as described above in Example 1), bis(triphenylphosphine)palladium(II) chloride (58.7 mg, 0.0836 mmol), ethyl 6-(4,4,5,5-tetramethyl-1,3,2-dioxaborolan-2-yl)-1H-indazole-3-carboxylate (260 mg, 0.84 mmol), aqueous sodium carbonate (2.00 M, 0.40 mL) and a mixture of dimethoxyethane:water:ethanol (50 mL, 7:3:2). The resulting mixture was subjected to microwave radiation at 140° C. for 5.0 minutes. A 20% mixture of methanol/dichloromethane (50 mL) was added, and the solution was filtered over celite and concentrated. Column chromatography purification (using 1:1 ethyl acetate/hexane followed by 3-5% methanol:dichloromethane) afforded 144 mg of ethyl 6-(6,7-dimethoxycinolin-4-yl)-1H-indazole-3-carboxylate as light yellow solid.

Step 2

A solution of potassium hydroxide in 85% methanol/water (2 M, 9 mL) was added to ethyl 6-(6,7-dimethoxycinolin-4-yl)-1H-indazole-3-carboxylate (125 mg, 0.33 mmol, prepared as described in Step 1 above) and the resulting mixture was stirred at room temperature for 12 h, then at 60° C. for 3 h. The pH of the mixture was adjusted to ˜3 using trifluoroacetic acid, and the solvent was removed in vacuo. The residue was diluted with methanol/dichloromethane (20%, 30 mL) and stirred for 1 hour resulting in the formation of two layers. The lower layer was separated and extracted again with methanol/dichloromethane (20%, 30 mL). The organics were combined and concentrated. The resulting residue was purified twice by column chromatography (using 5-30% methanol/dichloromethane) to afford 6-(6,7-dimethoxycinolin-4-yl)-1H-indazole-3-carboxylic acid as yellow solid.

Step 3

A mixture of 6-(6,7-dimethoxycinnolin-4-yl)-1H-indazole-3-carboxylic acid (30.0 mg, 0.0856 mmol, prepared as described in step 2 above), cyclopropylamine (0.012 mL, 0.17 mmol), N,N′-diisopropylcarbodiimide (21 μL), 1-hydroxybenzotriazole (6 mg, 0.04 mol), and N,N-dimethylformamide (4.0 mL) was stirred at room temperature for 18 hours. The solvent was evaporated and the residue was dissolved in ethyl acetate (50 mL) and washed with aqueous sodium bicarbonate (2×30 mL). The organic layer was concentrated and the product purified by column chromatography (using 3-10% methanol/ethyl acetate) followed by preparative HPLC to afford 11 mg (33% yield) of N-cyclopropyl-6-(6,7-dimethoxycinnolin-4-yl)-1H-indazole-3-carboxamide as a white solid. ¹H NMR (MeOD), 9.08 (s, 1H), 8.47 (d, J=7.2 Hz, 1H), 7.85 (s, 1H), 7.62 (s, 1H), 7.40 (d, J=7.2 Hz, 1H), 4.11 (s, 3H), 3.88 (s, 3H), 2.92 (m, 1H), 0.86 (m, 2H), 0.73 (m, 2H), LC/MS (EI), t_{R} 5.15 min (Method B), m/z 390.1 (M⁺+1).

### Example 8

### Synthesis of 6,7-dimethoxy-4-(5-morpholin-4-yl-1H-indol-1-yl)cinnoline hydroformate

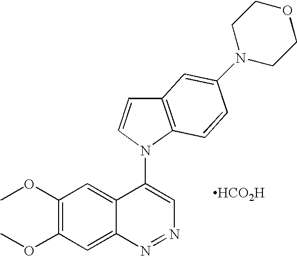 Step 1

Into a 5 mL microwave tube was added 4-bromo-6,7-dimethoxycinnoline (1000 mg, 3.716 mmol, prepared as described in Example 1 above), 5-bromoindole (871.9 mg, 4.447 mmol), copper(I) iodide (71 mg, 0.37 mmol), potassium carbonate (1.034 g, 7.479 mmol), N,N′-dimethyl-1,2-ethanediamine (160 μL, 1.5 mmol) and toluene (5 mL), and the resulting suspension was heated at 115° C. for 24 h. Volatiles were removed, and the residue was purified by preparative HPLC column chromatography to afford 70 mg of 4-(5-bromo-1H-indol-1-yl)-6,7-dimethoxyxinnoline.

Step 2

Into a 10 ml microwave tube was added 4-(5-bromo-1H-indol-1-yl)-6,7-dimethoxy-cinnoline (70 mg, 0.18 mmol, prepared as described in Step 1 above), morpholine (23.8 μL, 0.273 mmol), tetrahydrofuran (3.5 mL), tris(dibenzylideneacetone)dipalladium(0) (17 mg, 0.018 mmol), 9,9-dimethyl-4,5-bis(diphenylphosphino)xanthane (16 mg, 0.027 mmol) and sodium tert-butoxide (52.5 mg, 0.546 mmol) and the resulting suspension was heated at 70° C. for for 12 h. Volatiles were removed, and the residue was purified by preparative HPLC column chromatography (using a 10:90 to 80:20 gradient of acetonitrile:water (with 0.1% formic acid) and a flow rate of 45 mL/min) to afford 2 mg (3% yield) of 6,7-dimethoxy-4-(5-morpholin-4-yl-1H-indol-1-yl)cinnoline hydroformate. ¹H NMR (CDCl₃), δ 7.76 (s, 1H), 7.03 (s, 1H), 6.96 (s, 1H), 6.62 (m, 2H), 4.20 (m, 2H), 4.11 (s, 3H), 3.89 (s, 3H), 3.87 (m, 4H), 3.263 (m, 2H), 3.10 (m, 4H), LC/MS (EI), t_{R} 5.19 min (Method B), m/z 391 (M⁺+1).

### Example 9

### Synthesis of N-cyclopropyl-5-(6,7-dimethoxycinnolin-4-yl)-4,5,6,7-tetrahydro-1H-pyrazolo[4,3-c]pyridine-3-carboxamide

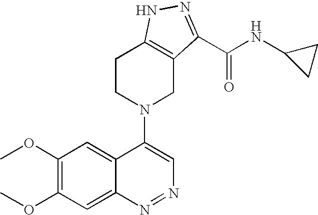 Step 1

To a solution of N,N-diisopropylamine (2.4 mL, 0.017 mol) in 20 mL of THF (20.0 mL, 0.246 mol) at 0° C. was added 2.0 M nBuLi in pentanes (8.5 mL). The reaction was stirred for 30 minutes at 0° C. and then cooled to −78° C. and a solution of 1-BOC-4-piperidone (3.20 g, 0.016 mol) in 20 mL of THF (20.0 mL, 0.246 mol) was added slowly. The mixture was stirred for 30 minutes at −78° C. and then a solution of diethyl oxalate (2.48 g, 0.017 mol) in THF (10.0 mL) was added in one portion. The mixture was stirred over night at room temperature. Water (200 mL) was added and the mixture was neutralized with 1 N HCl and extracted with 2×200 mL of EtOAc. The organic phase separated and washed with brine, dried (MgSO₄), filtered and concentrated under reduced pressure to provide crude tert-butyl 3-[ethoxy(oxo)acetyl]-4-oxopiperidine-1-carboxylate as a yellow oil used without further purification in step 2.

Step 2

A mixture of tert-butyl 3-[ethoxy(oxo)acetyl]-4-oxopiperidine-1-carboxylate (4.0 g, 0.013 mol) and acetic acid (8.0 mL, 0.141 mol) was treated drop-wise with hydrazine (1.0 mL, 0.032 mol) with stirring (note heat evolution). The mixture was stirred over night at room temperature and poured into an ice-cold saturated solution of NaHCO₃. The mixture was diluted with 50 mL of water and 50 mL of EtOAc. The organic fraction was washed with brine (25 mL), dried (MgSO₄) and concentrated to provide crude 5-tert-butyl 3-ethyl 1,4,6,7-tetrahydro-5H-pyrazolo[4,3-c]pyridine-3,5-dicarboxylate, which was used as such in step 3.

Step 3

A solution of 5-tert-butyl 3-ethyl 1,4,6,7-tetrahydro-5H-pyrazolo[4,3-c]pyridine-3,5-dicarboxylate (0.90 g, 0.0031 mol) in ethanol (30.0 mL) was treated with 5.0 M aqueous NaOH solution (10 mL). The reaction was stirred overnight at room temperature, diluted with 100 mL of water and washed with 2×50 mL of EtOAc. The aqueous fraction was acidified with 1.0 N aqueous HCL and extracted with 2×25 mL of EtOAc. The combined EtOAc extracts were washed with brine (25 mL), dried (MgSO₄) and concentrated to yield 5-(tert-butoxycarbonyl) 4,5,6,7-tetrahydro-1H-pyrazolo[4,3-c]pyridine-3-carboxylic acid as a white solid, which was used as such for step 4.

Step 4

5-(tert-Butoxycarbonyl) 4,5,6,7-tetrahydro-1H-pyrazolo[4,3-c]pyridine-3-carboxylic acid (40 mg, 0.15 mmol), cyclopropylamine (21 μL, 0.3 mmol), N,N′-diisopropylcarbodiimide (30 μL, 0.19 mmol), 1-hydroxybenzotriazole (10 mg, 0.07 mmol), N,N-dimethylformamide (0.3 mL) and methylene chloride (3.0 mL) were combined and stirred at room temperature for 5 h. The mixture was then concentrated and the residue was taken up in 50 mL of EtOAc, washed with 3×30 mL of NaHCO₃ and concentrated. The residue was purified by silica gel chromatography using a gradient elution going from 1% MeOH in 1:1 hexane:EtOAc to 3% MeOH in 1:1 hexanes:EtOAc to provide tert-butyl 3-[(cyclopropylamino)carbonyl]-1,4,6,7-tetrahydro-5H-pyrazolo[4,3-c]pyridine-5-carboxylate as a white solid. LC/MS (20/80 8 min, rt 5.6 min, M+H 307.2).

Step 5

tert-Butyl 3-[(cyclopropylamino)carbonyl]-1,4,6,7-tetrahydro-5H-pyrazolo[4,3-c]pyridine-5-carboxylate (0.034 g, 0.11 mmol), methylene chloride (2.0 mL) and trifluoroacetic acid (1.0 mL) were combined and stirred for 4 h at room temperature. The solvent was removed in vacuo and the residue was purified by trituration with ether to provide N-cyclopropyl-4,5,6,7-tetrahydro-1H-pyrazolo[4,3-c]pyridine-3-carboxamide trifluoroacetate salt as a white solid LC/MS (Method 20/80 8 min, rt 1.28 min, M+H 207.2).

Step 6

A mixture of 4-bromo-6,7-dimethoxycinnoline (0.010 g, 0.037 mmol), N-cyclopropyl-4,5,6,7-tetrahydro-1H-pyrazolo[4,3-c]pyridine-3-carboxamide trifluoroacetate (0.014 g, 0.046 mol), tris(dibenzylideneacetone)dipalladium(0) (3 mg, 0.004 mmol), N,N-dimethylacetamide (0.62 mL) and triethylamine (0.019 g, 0.18 mmol) was heated at 85° C. for 12 h. The solvent was removed in vacuo, and the residue was diluted with methanol/dichloromethane (5%, 1000 mL) and then filtered. The solution was washed with aqueous sodium bicarbonate. The organics were concentrated, and the residue was purified by preparative HPLC to afford 5.9 mg of N-cyclopropyl-5-(6,7-dimethoxycinnolin-4-yl)-4,5,6,7-tetrahydro-1H-pyrazolo[4,3-c]pyridine-3-carboxamide as a yellow solid. ¹H NMR (10% MeOD/CDCl₃), δ 8.56 (s, 1H), 7.55 (s, 1H), 7.12 (s, 1H), 4.88 (s, 2H), 4.02 (s, 3H), 4.01 (s, 3H), 3.84 (t, J=5.4 Hz, 2H), 3.00 (b, 2H), 2.74 (m, 1H), 0.77 (m, 2H), 0.54 (m, 2H). LC/MS (EI), t_{R} 2.77 min (Method B), m/z 395.1 (M⁺+1).

### Example 10

### Synthesis of 4-[1-(4-fluorobenzyl)-1H-pyrazol-4-yl]-6,7-dimethoxycinnoline hydroformate

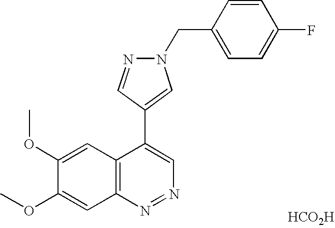 Step 1

Into a microwave tube was added 4-bromo-6,7-dimethoxycinnoline (200 mg, 0.8 mmol, prepared as described in Example 1 above), bis(triphenylphosphine) palladium(II) chloride (95.6 mg, 0.136 mmol), tert-butyl-4,-(4,4,5,5-tetramethyl-1,3,2-dioxaborolan-2-yl)- 1H-pyrazole-1-carboxylate (200 mg, 0.0008 mol), aqueous sodium carbonate (2.00 M, 0.28 mL) and a mixture of dimethoxyethane:water:ethanol (5 mL, 7:3:2). The resulting suspension was subjected to microwave radiation at 140° C. for 600 seconds. The reaction was filtered through celite, which was washed with methanol. Concentration, followed by ISCO chromatographic purification (using a gradient of 50% ethyl acetate:hexanes to 100% ethyl acetate, followed by elution with a 70:30:1 mixture of ethyl acetate:methanol:ammonia afforded 140 mg (70% yield) of 6,7-dimethoxy-4-(1H-pyrazol-4-yl)cinnoline as a yellow solid.

Step 2

Sodium hydride (5 mg, 0.2 mmol) was added to dimethylformamide (2 mL) in a flame-dried round bottom flask under an atmosphere of nitrogen. 6,7-dimethoxy-4-(1H-pyrazol-4-yl)cinnoline (25 mg, 0.098 mmol, prepared as described above in Step 1) was added and the reaction stirred at room temperature for 1 h. A solution of α-bromo-4-fluorotoluene (60 mg, 0.0003 mol) in dimethylformamide (0.5 mL) (prepared under a nitrogen atmosphere) was then added, and the resulting mixture was stirred at room temperature for 16 h. The mixture was concentrated, and the residue purified by preparative HPLC (using a gradient of 20-80% acetonitrile with 0.1% formic acid). Further purification by column chromatography (using 7 M ammonia in methanol as the eluent) afforded 6 mg of 4-[1-(4-fluorobenzyl)-1H-pyrazol-4-yl]-6,7-dimethoxycinnoline hydroformate. ¹H NMR (CDCl₃), d 9.07 (s, 1H), 7.98 (s, 1H), 7.81 (s,1H), 7.80 (s, 1H), 7.39-7.35 (m, 2H), 7.31 (m, 2H), 7.15-7.09 (m, 2H), 5.43 (s, 2H), 4.13 (s, 3H), 4.00 (s, 3H), LC/MS (EI), t_{R} 5.3 min (Method A), m/z 365 (M⁺+1).

The following compounds were prepared in a similar manner to Example 10 using different starting materials.

### 6,7-Dimethoxy-4-[1-(4-methylbenzyl)-1H-pyrazol-4-yl]cinnoline

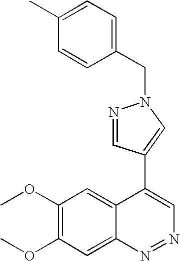

Prepared using 1-bromoethyl-4-methylbenzene in Example 10, Step 2 to give 4.6 mg of above compound. LC/MS (EI), t_{R} 6.48 min (Method A), m/z 361 (M⁺+1).

### 4-[1-(4-tert-Butylbenzyl)-1H-pyrazol-4-yl]-6,7-dimethoxycinnoline

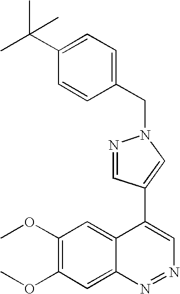

Prepared using 4-tert-butylbenzyl bromide in Example 10, Step 2 to give 3.2 ve compound. LC/MS (EI), t_{R} 7.80 min (Method A), m/z 403 (M⁺+1).

### 4-[1-(Biphenyl-4-ylmethyl)-1H-pyrazol-4-yl]-6,7-dimethoxycinnoline

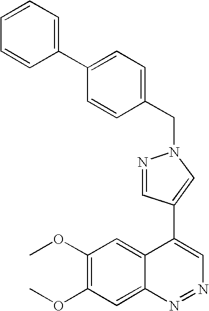

Prepared using 4-(bromophenyl)biphenyl in Example 10, Step 2 to give 4.9 ve compound. LC/MS (EI), t_{R} 7.70 min (Method A), m/z 423 (M⁺+1).

### Methyl 4-{[4-(6,7-dimethoxycinnolin-4-yl)-1H-pyrazol-1-yl]methyl}benzoate

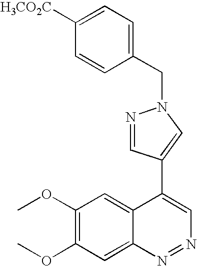

Prepared using methyl 4-bromomethylbenzoate in Example 10, Step 2 to give 3.7 mg (of above compound. LC/MS (EI), t_{R} 5.99 min (Method A), m/z 405 (M⁺+1).

### 4-[1-(Biphenyl-2-ylmethyl)-1H-pyrazol-4-yl]-6,7-dimethoxycinnoline

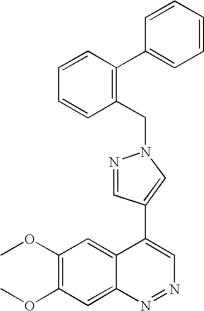

Prepared using 2-phenylbenzyl bromide in Example 10, Step 2 to give 9.6 mg of above compound. LC/MS (EI), t_{R} 7.62 min (Method A), m/z 423 (M⁺+1).

### 6,7-Dimethoxy-4-{1-[3-(trifluoromethyl)benzyl]-1H-pyrazol-4-yl}cinnoline

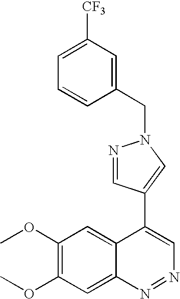

Prepared using 3-(trifluoromethyl)benzyl bromide in Example 10, Step 2 to give above compound. LC/MS (EI), t_{R} 6.88 min (Method A), m/z 415 (M⁺+1).

### 6,7-Dimethoxy-4-{1-[2-(trifluoromethyl)benzyl]-1H-pyrazol-4-yl}cinnoline

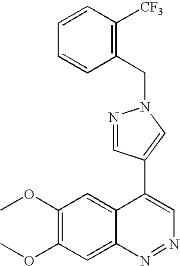

Prepared using 2-(trifluoromethyl)benzyl bromide in Example 10, Step 2 to give 2.6 mg of above compound. LC/MS (EI), t_{R} 6.92 min (Method A), m/z 415 (M⁺+1).

### 2-{[4-(6,7-Dimethoxycinnolin-4-yl)-1H-pyrazol-1-yl]methyl}benzonitrile

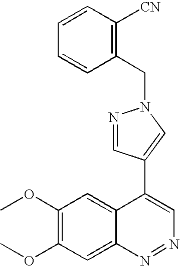

Prepared using 2-cyanobenzyl bromide in Example 10, Step 2 to give 8.9 mg of above compound. LC/MS (EI), t_{R} 5.8 min (Method A), m/z 372 (M⁺+1).

### 6,7-Dimethoxy-4-[1-(3-methylbenzyl)-1H-pyrazol-4-yl]cinnoline

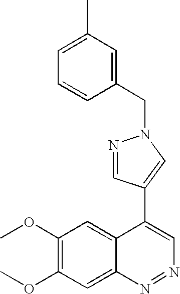

Prepared using 1-bromomethyl-3-methyl benzene in Example 10, Step 2 to give 1.4 mg of above compound. LC/MS (EI), t_{R} 6.98min (Method A), m/z 361 (M⁺+1).

### 4-{[4-(6,7-Dimethoxycinnolin-4-yl)-1H-pyrazol-1-yl]methyl}benzonitrile

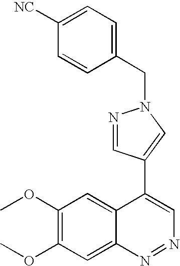

Prepared using 4-cyanobenzyl bromide in Example 10, Step 2 to give above compound. LC/MS (EI), t_{R} 5.81 min (Method A), m/z 372 (M⁺+1).

### 6,7-Dimethoxy-4-[1-(2-methylbenzyl)-1H-pyrazol-4-yl]cinnoline

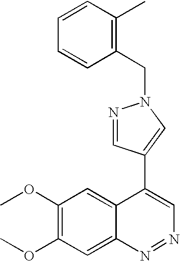

Prepared using 1-(bromomethyl)-2-methylbenzene in Example 10, Step 2 to give 8.7 mg of above compound. LC/MS (EI), t_{R} 6.42 min (Method A), m/z 361 (M⁺+1).

### Example 11

### Synthesis of 6,7-dimethoxy-4-{1-[4-(trifluoromethoxy)benzyl]-1H-pyrazol-4-yl}cinnoline

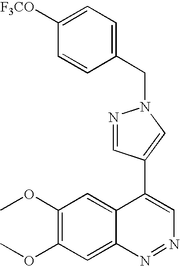

Into a flame-dried 10 mL round bottom flask under nitrogen was added sodium hydride (5 mg, 0.20 mmol), 2 mL of DMF and 6,7-dimethoxy-4-(1H-pyrazol-4-yl)cinnoline (25 mg, 0.098 mmol). The reaction was stirred for 1 h at room temperature followed by the addition via cannula of a solution of 4-(trifluoromethoxy)benzyl bromide (70 mg, 0.30 mmol) in 0.5 mL of DMF, which was prepared in a flame-dried 10 mL round bottom flask under nitrogen. The reaction color turned from light brown to dark red and stirring continued at 25° C. for 16 h. The reaction mixture was assayed by LCMS and showed product. The reaction was concentrated and the residue was purified by preparative TLC with 50% EtOAc:Hex followed by 100% EtOAc:Hex in two separate batches to give a total of 6 mg of 6,7-dimethoxy-4-{1-[4-(trifluoromethoxy)benzyl]-1H-pyrazol-4-yl}cinnoline.

### Example 12

### Synthesis of 6-(6,7-dimethoxycinnolin-4-yl)-N,N-diethyl-1H-indazole-3-carboxamide

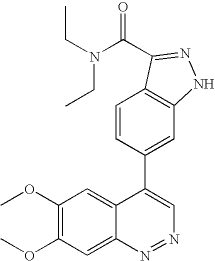 Step 1

Into a 10 mL microwave tube was added 4-bromo-6,7-dimethoxycinnoline (150 mg, 0.56 mmol), bis(triphenylphosphine)palladium(II) chloride (58.7 mg, 0.0836 mmol), ethyl 6-(4,4,5,5-tetramethyl-1,3,2-dioxaborolan-2-yl)-1H-indazole-3-carboxylate (260 mg, 0.84 mmol), 2.00 M sodium carbonate in water (0.40 mL) and DME:Water:EtOH=7:3:2 (7:3:2, 1,2-Dimethoxyethane:Water:Ethanol, 5.01 mL). The reaction was irradiated in a microwave reactor at 300 watts to 140° C. for 10 minutes. The reaction mixture was diluted with 50 mL of 20% MeOH/DCM and filtered over celite. The organic solution was concentrated and purified by column chromatography (1:1 EtOAc/hexane 3 cv followed by 3-5% MeOH/DCM) to give ethyl 6-(6,7-dimethoxycinnolin-4-yl)-1H-indazole-3-carboxylate as a light yellow solid.

Step 2

Ethyl 6-(6,7-dimethoxycinnolin-4-yl)-1H-indazole-3-carboxylate was treated with 9 mL of 2M KOH in 85% MeOH/water at 25° C. for 12 hours and then warmed to 60° C. for 3 hours. The solution was adjusted to a pH of about 3 by the careful addition of trifluoroacetic acid and then the solvent was evaporated under vacuum. The residue was diluted with 30 mL of 20% MeOH/DCM and kept stirring for 1 hour to form two layers and separated. The bottom layer was extracted with 20% MeOH/DCM (30 mL) and the combined DCM solutions were concentrated. The resulting residue was purified by column chromatography using a gradient elution going from 5% to 30% MeOH in DCM to give 6-(6,7-dimethoxycinnolin-4-yl)-1H-indazole-3-carboxylic acid as a yellow solid.

Step 3

6-(6,7-Dimethoxycinnolin-4-yl)-1H-indazole-3-carboxylic acid (30.0 mg, 0.0856 mmol), N-ethylethanamine (0.030 mL, 0.29 mmol), N,N′-diisopropylcarbodiimide (18 μL, 0.11 mmol), 1-hydroxybenzotriazole (5 mg, 0.04 mmol) and 2.5 mL of DMF were combined and stirred at 25° C. for 24 hours. The solvent was then evaporated and the residue was dissolved in 40 mL of DCM and washed with 1% sodium bicarbonate. The organic phase was concentrated and purified by HPLC (prep0680, rt 5.25 min) to give 6-(6,7-dimethoxycinnolin-4-yl)-N,N-diethyl-1H-indazole-3-carboxamide as a light yellow solid. LC/MS 2080_{—}8 min, retention time 5.16 min, M+H 406.1.

### Example 12

### Synthesis of 6,7-dimethoxy-4-[2-(4-methylpiperazin-1-yl)pyrimidin-5-yl]cinnoline

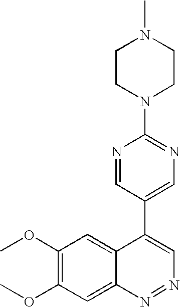

Into a 5 mL microwave tube was added 4-bromo-6,7-dimethoxycinnoline (50.0 mg, 0.186 mmol), 2-(4-methylpiperazin-1-yl)-5-(4,4,5,5-tetramethyl-1,3,2-dioxaborolan-2-yl)pyrimidine (145 mg, 0.478 mmol), bis(triphenylphosphine)palladium(II) chloride (26.9 mg, 0.0384 mmol), 2.00 M sodium carbonate in water (139 uL) and DME:Water:EtOH=7:3:2 (7:3:2, 1,2-Dimethoxyethane:Water:Ethanol, 895 uL). The cloudy, brown suspension was irradiated in a microwave at 300 W to 140° C. for 5.0 minutes. The reaction mixture was filtered through a celite plug and washed with methanol. The solution was concentrated under reduced pressure and the remaining residue was purified by rotary chromatography using a gradient elution going from 100% chloroform to 10% methanol in chloroform to provide 64 mg of 6,7-dimethoxy-4-[2-(4-methylpiperazin-1-yl)pyrimidin-5-yl]cinnoline.

### Example 13

### Synthesis of 5-(6,7-dimethoxycinnolin-4-yl)-N-(pyridin-3-ylmethyl)pyridin-2-amine trifluoroacetic acid salt

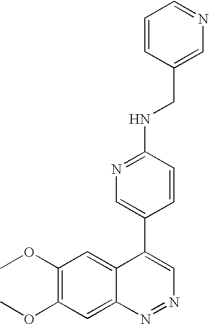

A mixture of 4-(6-fluoropyridin-3-yl)-6,7-dimethoxycinnoline (0.050 g, 0.18 mmol), 3-(aminomethyl)pyridine (0.038 g, 0.35 mmol), and DMSO (1 mL) was heated in an oil bath at 120° C. for 16 h. The resulting solution was purified by preparative HPLC (10-90% CH₃CN/H2O modified with 0.1% TFA) to give 5-(6,7-dimethoxycinnolin-4-yl)-N-(pyridin-3-ylmethyl)pyridin-2-amine trifluoroacetic acid salt as a red solid. MH+ theoretical value 374: Observed value 374.

### Example 14

### Synthesis of 1-(4-(6,7-dimethoxycinnolin-4-yl)benzyl)azetidine-3-carboxylic acid

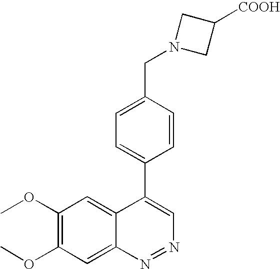 Step 1

A mixture of 4-bromo-6,7-dimethoxycinnoline (0.1 g, 0.4 mmol), 4-formylphenylboronic acid (0.06 g, 0.4 mmol), palladium tetrakis-triphenylphosphine (0.02 g, 0.02 mmol), cesium carbonate (0.3 g, 1 mmol), and water (2 mL) were combined into a sealed tube under nitrogen atmosphere. After overnight heating at 80° C., LC/MS showed complete conversion. The reaction mixture was allowed to cool to room temperature and concentrated to give 4-(6,7-dimethoxycinnolin-4-yl)benzaldehyde which was used in the next step with out further purification.

Step 2

To a solution of 4-(6,7-dimethoxycinnolin-4-yl)benzaldehyde (0.1 g, 0.4 mmol) and 3-azetidinecarboxylic acid (0.04 g, 0.4 mmol) in dichloromethane was added sodium triacetoxyborohydride (0.1 g, 0.5 mmol) and trifluoroacetic acid (0.05 g, 0.4 mmol) at room temperature. LC/MS showed partial conversion after overnight stirring at room temperature. More sodium triacetoxyborohydride was added and stirring was continued for another few hours until LC/MS showed full conversion. The product appeared to be very polar based on LC retention time. Purification of the crude product was carried out by prep-plate TLC (10% MeOH/DCM). The isolated rich cut still contained some impurity and was purified again via shimadzu HPLC to recover the TFA salt of desired product as yellow oil. MS (ESI, pos. ion) m/z: 380.0 (M+1).

### Example 14

### Synthesis of 6,7-dimethoxy-4-(6-(1,2,3,6-tetrahydropyridin-4-yl)pyridin-3-yl)cinnoline

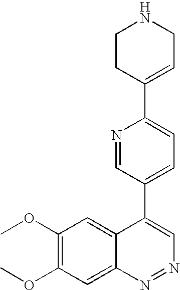 Step 1

Into a suspension of 4-bromo-6,7-dimethoxycinnoline (0.50 g, 1.9 mmol), 2-chloropyridine-5-boronic acid (0.29 g, 1.9 mmol), and disodium carbonate monohydrate (0.35 mg, 2.8 mmol) in a mixed solvent of DME (3 mL), EtOH (1.8 mL) and water (1.5 mL) was bubbled N₂ for 5 min. Then dichlorobis(triphenylphosphine)palladium(II) (0.13 g, 0.19 mmol) was added and the reaction mixture was heated at 90° C. for 3 h. The reaction mixture was cooled to room temperature, diluted with EtOAc and water and the product was isolated by filtration. The solid collected was washed with a small amount of EtOAc and ether, dried in a vacuum oven to give 4-(6-chloropyridin-3-yl)-6,7-dimethoxycinnoline as light-yellow solid.

Step 2

A mixture of 4-(6-chloropyridin-3-yl)-6,7-dimethoxycinnoline (0.12 g, 0.4 mmol), tert-butyl 4-(4,4,5,5-tetramethyl-1,3,2-dioxaborolan-2-yl)-5,6-dihydropyridine-1(2H)-carboxylate (0.18 g, 0.6 mmol), and tetrakis(triphenylphosphine)palladium (0.023 g, 0.02 mmol) in dioxane (1 mL) was treated with 2M aqueous solution of potassium carbonate (0.16 g, 1.2 mmol). The reaction mixture was heated at 100° C. for 2 h. After cooling to room temperature, the reaction mixture was diluted with EtOAc and saturated NH₄Cl and transferred to a separatory funnel. The layers were separated and the aqueous phase was extracted with EtOAc. The combined organics were washed with brine, dried over Na₂SO₄, filtered and concentrated. The crude product was chromatographed through a Redi-Sep® pre-packed silica gel column (40 g), eluting with a gradient of 0% to 5% MeOH in DCM, to provide tert-butyl 4-(5-(6,7-dimethoxycinnolin-4-yl)pyridin-2-yl)-5,6-dihydropyridine-1(2H)-carboxylate) as a light-yellow solid.

Step 3

To tert-butyl 4-(5-(6,7-dimethoxycinnolin-4-yl)pyridin-2-yl)-5 ,6-dihydropyridine-1(2H)-carboxylate (72 mg, 0.16 mmol) dissolved in DCM (1 mL) was added TFA (0.3 ml, 3.9 mmol). The reaction mixture was stirred at RT under nitrogen for 1 h. The solvent was removed in vacuo and the residue was partitioned between DCM and saturated NaHCO₃. The aqueous fraction was back extracted with DCM and the combined organics were dried (Na₂SO₄) and concentrated. Trituation with ether gave 6,7-dimethoxy-4-(6-(1,2,3,6-tetrahydropyridin-4-yl)pyridin-3-yl)cinnoline as a light-yellow solid. MS (ESI, pos. ion) m/z: 349 (M+1).

### Example 15

### Synthesis of 4-(6-(cyclopropylmethoxy)pyridin-3-yl)-6,7-dimethoxycinnoline

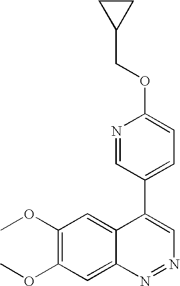

To the suspension of 4-bromo-6,7-dimethoxycinnoline (70 mg, 0.26 mmol), 2-(cyclopropylmethoxy)-5-(4,4,5,5-tetramethyl-1,3,2-dioxaborolan-2-yl)pyridine (75 mg, 0.27 mmol), and disodium carbonate monohydrate (48 mg, 0.39 mmol) in a mixed solvent of DME (0.5 mL), EtOH (0.3 mL) and water (0.25 mL) was bubbled N₂ for 5 min. Then dichlorobis(triphenylphosphine)palladium(II) (18 mg, 0.026 mmol) was added and the reaction mixture was heated at 90° C. for 2 h. The reaction mixture was cooled to room temperature, diluted with EtOAc and water, and transferred to a separatory funnel. The layers were separated and the aqueous was extracted with EtOAc. The combined organics were washed with brine, dried over Na₂SO₄, filtered and concentrated. The crude product was chromatographed through a Redi-Sep® pre-packed silica gel column (12 g), eluting with a gradient of 0% to 5% MeOH in DCM, followed by washing with ether (15 mL) to provide 4-(6-(cyclopropylmethoxy)-pyridin-3-yl)-6,7-dimethoxycinnoline as a light-yellow solid. MS (ESI, pos. ion) m/z: 338 (M+1).

### Example 16

### Synthesis of 6,7-dimethoxy-4-(4-(oxazol-2-yl)phenyl)cinnoline

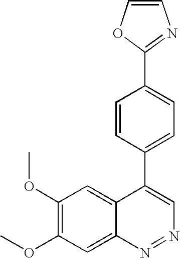

A mixture of 2-(4-(4,4,5,5-tetramethyl-1,3,2-dioxaborolan-2-yl)phenyl)oxazole (0.089 g, 0.33 mmol), 4-bromo-6,7-dimethoxycinnoline (0.080 g, 0.3 mmol), palladium tetrakis-triphenylphosphine (0.017 g, 0.015 mmol), cesium carbonate (0.26 g, 0.80 mmol), and water (2.4 mL) were added to a sealed tube under atmosphere of N₂. The resulting mixture was heated to 80° C. over the weekend. LC/MS showed complete conversion. The reaction mixture was filtered over a cake of celite and then rinsed with MeOH. Purification with Biotage (MeOH/DCM) produced a rich cut that still contained impurities. Purified two more times using prep-plate TLC (5% MeOH/DCM) to provide the product as slight yellow solid. MS (ESI, pos. ion) m/z: 334 (M+1)

### Example 17

### Synthesis of 6-(6,7-dimethoxycinnolin-4-yl)-N-isopropylbenzo[d]isothiazole-3-carboxamide

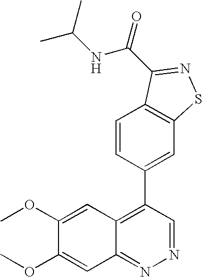 Step 1

A solution of 3-bromobenzenethiol (6.00 g, 31.7 mmol) in CH₂Cl₂ (16 mL) was added slowly dropwise to neat oxalyl chloride (13.8 mL, 159 mmol) at room temperature with stirring. The resultant mixture was heated to reflux and stirred overnight at which point LCMS analysis indicated the reaction was complete. The reaction mixture was then cooled to room temperature and the volatiles were removed in vacuo. A yellow solid was obtained which was S-3-bromophenyl-2-chloro-2-oxoethanethioate.

Step 2

Aluminum chloride (12.9 g, 96.6 mmol) was stirred at room temperature in carbon disulfide (10.8 ml) until all the solids were suspended. A suspension of S-3-bromophenyl-2-chloro-2-oxoethanethioate (6.00 g, 21.5 mmol) in carbon disulfide (10.8 mL, 2M) was then added very slowly dropwise to the AlCl₃ suspension. The flask was then equipped with a reflux condenser and the reaction mixture was heated to 45° C. for 2 hrs. LCMS analysis indicated complete consumption of the starting material. The reaction mixture was cooled to room temperature and the supernatant was poured into ice water. Water was then added to the solids remaining in the flask (Caution: very exothermic!) and diethyl ether was added. The resultant orange precipitate was poured into ice water and filtered to obtain an orange solid which was dried overnight to give 6-bromobenzo[b]thiophene-2,3-dione.

Step 3

Ammonium hydroxide (28% aqueous solution) (3.91 mL, 28.4 mmol) was added slowly dropwise to a solution of 6-bromobenzo[b]thiophene-2,3-dione (300 mg, 1.23 mmol) in MeOH (2 ml) cooled to 10 C, maintaining the temperature between 10-20° C. The ice bath was removed and the resultant mixture was stirred overnight at room temperature after which time the reaction mixture was re-cooled to 10 C and hydrogen peroxide (30%) (0.391 mL, 3.83 mmol) was added slowly dropwise. The ice bath was removed and the reaction mixture was stirred at room temperature for 1 hour. The resulting precipitate was filtered and washed with water. After air-drying, a light tan solid was obtained which was 6-bromobenzo[d]isothiazole-3-carboxamide.

Step 4

A suspension of 6-bromobenzo[d]isothiazole-3-carboxamide (274 mg, 1066 μmol) in EtOH (5.9 mL) and 6N sodium hydroxide (356 μL, 2135 μmol) was heated to reflux for 2 hrs. LCMS analysis indicated complete conversion to the acid. The reaction mixture was cooled to room temperature, acidified with 1N HCl, and extracted with ethyl acetate. The combined organics were washed with brine, dried over MgSO₄, filtered and concentrated to give 6-bromobenzo[d]isothiazole-3-carboxylic acid which was used without further purification.

Step 5

Sulfuryl dichloride (86.7 mg, 728 μmol) was added to a solution of 6-bromobenzo[d]-isothiazole-3-carboxylic acid (188 mg, 728 μmol). The reaction mixture was stirred for 30 min before removing the volatiles by rotovap. The residue was taken up in CH₂Cl₂ (0.587 ml) and a solution of 2-propylamine (62.5 μL, 728 μmol) and triethylamine (101 μl, 728 μmol) in CH₂Cl₂ (1.2 ml) was added. The reaction mixture was stirred at room temperature until LCMS analysis indicated complete conversion to the desired product. The reaction mixture was diluted with distilled water and ethyl acetate. The layers were separated and the aqueous was extracted with ethyl acetate. The combined organics were washed with brine and dried over Na₂SO₄, filtered and concentrated to give 6-bromo-N-isopropylbenzo[d]isothiazole-3-carboxamide.

Step 6

A solution of 6-bromo-N-isopropylbenzo[d]isothiazole-3-carboxamide (200 mg, 668 μmol), 4,4,5,5-tetramethyl-2-(4,4,5,5-tetramethyl-1,3,2-dioxaborolan-2-yl)-1,3,2-dioxaborolane (204 mg, 802 μmol), potassium acetate (131 mg, 1337 μmol), and dichloropalladiumbis-(diphenylphosphinoferrocene) (34 mg, 47 μmol) in dioxane (3.2 mL) was heated to 130 C overnight after which time LCMS analysis indicated complete conversion to the desired product. The reaction mixture was filtered through celite give a brown solid. Purification was performed by Biotage pre-packed silica gel column (25M) using a gradient of 12-100% ethyl acetate/hexanes to give N-isopropyl-6-(4,4,5,5- tetramethyl-1,3,2-dioxaborolan-2-yl)benzo[d]isothiazole-3-carboxamide.

Step 7

To a solution of N-isopropyl-6-(4,4,5,5-tetramethyl-1,3,2-dioxaborolan-2-yl)benzo[d]-isothiazole-3-carboxamide (69 mg, 199 μmol) in DME (2.4 mL) was added 4-bromo-6,7-dimethoxycinnoline (54 mg, 199 μmol), bis(triphenylphosphine)palladium (II) chloride (7.0 mg, 10.0 μmol) followed by an aqueous solution of cesium carbonate (175 mg, 538 μmol) (1 ml H20). The reaction mixture was heated to 80° C. for two hours before sampling by LCMS which indicated the reaction was complete. The reaction mixture was cooled to room temperature, diluted with distilled water and ethyl acetate. The layers were separated and the aqueous was extracted with ethyl acetate. The combined organics were washed with brine, dried over Na₂SO₄, filtered and concentrated. The residue was purified by Biotage, 25M column, 12-100% ethyl acetate (10% MeOH)/hexanes to give 6-(6,7-dimethoxycinnolin-4-yl)-N-isopropylbenzo[d]isothiazole-3-carboxamide.

### Example 18

### Synthesis of 4-(6-(3,3-difluoroazetidin-1-yl)pyridin-3-yl)-6,7-dimethoxycinnoline

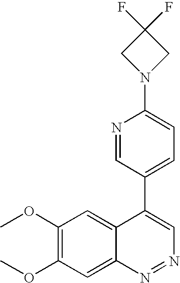 Step 1

To a 250 mL round-bottomed flask was added 4-bromo-6,7-dimethoxycinnoline (4.0064 g, 14.89 mmol) and tetrakis(triphenylphosphine)palladium (0) (0.8667 g, 0.7444 mmol) in 250 mL 1,2-dimethoxyethane. 6-Fluoropyridin-3-ylboronic acid (0.2849 g, 1.983 mmol) was added, followed by an aqueous solution of cesium carbonate (1.6792 g, 4.868 mmol) (10 mL water), and the reaction mixture was stirred at 80° C. for 3 hours. The reaction mixture was allowed to cool to room temperature. The solution was placed in a separatory funnel and deionized water and ethyl acetate was added. The aqueous layer was extracted with ethyl acetate. The combined organic layers were washed with water, brine, dried with MgSO₄, filtered, and concentrated. The tan solid was taken up in ether and allowed to stir for 15 minutes. The solid was then filtered and dried by vacuum to produce 4-(6-fluoropyridin-3-yl)-6,7-dimethoxycinnoline (3.15 g).

Step 2

In a microwave vial was placed 4-(6-fluoropyridin-3-yl)-6,7-dimethoxycinnoline (0.0621 g, 0.218 mmol) and potassium carbonate (0.3126 g, 2.22 mmol) in 2 mL DMSO. 3,3-Difluoroazetidine hydrochloride (0.2799 g, 2.18 mmol) was added and the temperature was brought to 90° C. to stir overnight. The reaction solution was allowed to cool to room temperature. The solution was moved to a separatory funnel and deionized water and ethyl acetate was added. The aqueous layer was extracted with ethyl acetate. The combined organic layers were washed with water, brine, dried with MgSO₄, filtered, and concentrated to produce 4-(6-(3,3-difluoroazetidin-1-yl)pyridin-3-yl)-6,7-dimethoxycinnoline (70 mg).

### Example 19

### Synthesis of provide 4-(5-(6,7-dimethoxycinnolin-4-yl)pyridin-2-yl)-1-methylpiperazin-2-one

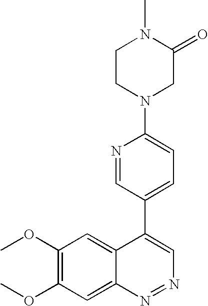

In a microwave vial was placed 4-(6-fluoropyridin-3-yl)-6,7-dimethoxycinnoline (0.0652 g, 0.229 mmol) in 2 ml DMSO. 1-Methylpiperazin-2-one hydrochloride (0.3626 g, 2.29 mmol) and potassium carbonate (0.147 ml, 2.40 mmol) was added and the temperature was brought to 90° C. to stir overnight. The reaction solution was allowed to cool to room temperature. The solution was moved to a separatory funnel and deionized water and ethyl acetate were added. The aqueous layer was extracted with ethyl acetate. The combined organic layers were washed with water, brine, dried with MgSO₄, filtered, and concentrated. The crude product was adsorbed onto a plug of silica gel and chromatographed through a Biotage pre-packed silica gel column (25M), eluting with a gradient of 1% to 5% MeOH in CH₂Cl₂, to provide 4-(5-(6,7-dimethoxycinnolin-4-yl)-pyridin-2-yl)-1-methylpiperazin-2-one (0.0413 g).

### Biological Examples

### Example 20

### mPDE10A7 Enzyme Activity and Inhibition

Enzyme Activity:

To analyze the enzyme activity, 5 μL of serial diluted mPDE10A7 containing lysate were incubated with equal volumes of diluted (100-fold) fluorescein labeled cAMP or cGMP for 30 minutes in MDC HE 96-well assay plates at room temperature. Both the enzyme and the substrates were diluted in the following assay buffer: Tris/HCl (pH 8.0) 50 mM, MgCl₂ 5 mM, 2-mercaptoethanol 4 mM, BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP Assay kit (Molecular Device) was used to assess enzyme properties of mmPDE10A7. Data were analyzed with SoftMax Pro.

Enzyme Inhibition:

To check the inhibition profile, 10 μL of serial diluted compounds were incubated with 30 μl of diluted PDE enzymes in a 96-well polystyrene assay plate for 30 minutes at room temperature. After incubation, 5 μL of the compound-enzyme mixture were aliquoted into a MDC HE black plate, mixed with 5 μL of 100-fold diluted fluorescein labeled substrates (cAMP or cGMP), and incubated for 30 minutes at room temperature. The reaction was stopped by adding 20 μL of diluted binding reagents and counted in an Analyst GT for fluorescence polarization. The data were analyzed with SoftMax Pro. Compounds of the invention inhibited the mPDE10A7 enzyme.

The IC₅₀ values of representative compounds of this invention is shown in Table 2 below.

TABLE 2

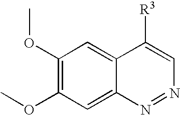

| | | mPDE10A7, |
|---|---|---|
| Cpd | | IC₅₀ |
| # | R³ | (nM) |
| 1 | 3-morpholin-4-ylphenyl | 57.88 |
| 3 | 3-ethylsulfonylphenyl | 29.81 |
| 4 | 6-(piperidin-1-yl)-3,4-dihydroisoquinolin-1(2H)-one | 166.76 |
| 5 | 3-(1-methyl-1H-pyrazol-4-yl)phenyl | 79.82 |
| 7 | 3-(cyclopropylaminocarbonyl)-4,5,6,7-tetrahydro- | 19.28 |
| | 1H-pyrazolo[4,3-c]-pyridin-5-yl | |
| 8 | 3-(4,4-dimethyl-4,5-dihydro 1,3-oxazol-2-yl)phenyl | 30.59 |
| 10 | 1-(3-methoxyphenyl)-1H-pyrazol-4-yl | 68 |
| 12 | 3-(ethoxycarbonyl)-1H-indazol-5-yl | 12.1 |
| 14 | 3-acetylaminophenyl | 42.96 |
| 15 | 3-dimethylaminophenyl | 22.83 |
| 20 | 5-(morpholin-4-yl)indol-1-yl | 209.08 |
| 21 | 1-(4-methylbenzyl)-1H-pyrazol-4-yl | 43.56 |
| 23 | 1-(4-phenylbenzyl)-1H-pyrazol-4-yl | 473.95 |
| 24 | 1-(4-methoxycarbonylbenzyl)-1H-pyrazol-4-yl | 48.7 |
| 26 | 1-(3-trifluoromethylbenzyl)-1H-pyrazol-4-yl | 181.82 |
| 28 | 1-(2-cyanobenzyl)-1H-pyrazol-4-yl | 101.34 |
| 32 | 1-(4-trifluoromethoxybenzyl)pyrazol-4-yl | 458.89 |
| 33 | 6-(morpholin-4-yl)pyridin-3-yl | 11.06 |
| 35 | 1-(4-fluorobenzyl)-1H-pyrazol-4-yl | 10.39 |
| 42 | 3-fluoro-2-morpholin-4-ylpyridin-4-yl | 62.01 |
| 43 | 2-(1-methylpiperazin-4-yl)pyrimidin-5-yl | 19.86 |
| 46 | 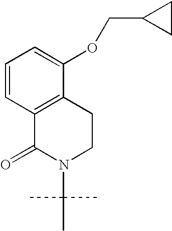 | 246.72 |
| 52 | 3-ethoxycarbonylbenzo[d]isoxazol-5-yl | 48.45 |
| 54 | 2-(2-oxo-1-methylpiperazin-4-yl)pyridin-4-yl | 55.79 |
| 55 | 2-(3-methoxypyrrolidin-1-yl)pyridin-5-yl | 10.54 |
| 56 | 2-(4,4-difluoropiperidin-1-yl)pyridin-5-yl | 29.54 |
| 59 | 2-(isopropylamino)pyridin-5-yl | 8.23 |
| 62 | 2-(2-methoxyethylamino)pyridin-5-yl | 36.18 |
| 63 | 2-(2-aminoethylamino)pyridin-5-yl | 613.6 |
| 65 | 2-(1-tert-butoxycarbonylazetidin-4- | 66.64 |
| | ylmethylamino)pyridin-5-yl | |
| 68 | 4-(quinolin-2-ylmethyloxy)phenyl | 106.3 |
| 72 | 2-(3-(trifluoromethyl)-5,6,7,8-tetrahydro- | 68.56 |
| | [1,2,4]triazolo[4,3-a]pyrazin-7-yl)pyridin-5-yl | |
| 79 | 2-(4-methylaminopiperidin-1-yl)pyridin-5-yl | 361.87 |
| 81 | 2-(phenylamino)pyridin-5-yl | 89.08 |
| 87 | 2-(phenoxy)pyridin-5-yl | 151.67 |
| 88 | 2-(4-dimethylaminophenylamino)pyridin-5-yl | 178.98 |
| 89 | 2-(cyclopropylmethyloxy)pyridin-5-yl | 33.79 |
| 93 | 2-(2-pyridin-2-ylethylamino)pyridin-5-yl | 17.93 |
| 95 | 2-(3-cyanophenylamino)pyridin-5-yl | 87.9 |
| 100 | 3-fluoro-4-acetylphenyl | 254.32 |
| 101 | 4-(4-carboxyazetidin-1-yl)phenyl | 231.36 |
| 108 | 2-(4-hydroxy-4-phenylpiperidin-1-yl)pyridin-5-yl | 21.43 |
| 115 | 3-fluoro-2-[3-fluoro-2-(isopropylamino)pyridin-5- | 676.31 |
| | yl]pyridin-5-yl | |
| 118 | 2-(4-(oxazol-5-yl)phenylamino)pyridin-5-yl | 38.58 |
| 119 | 2-[5-(cyclopropyl)-[1.3.4]-thiadiazol-2- | 258.74 |
| | ylamino]pyridin-5-yl | |
| 122 | 2-(3-trifluoromethoxybenzylamino)pyridin-5-yl | 185.92 |
| 123 | 6-isopropylamino-2-methylpyridin-3-yl | 18.55 |
| 142 | 2-(3-trifluoromethoxybenzylamino)pyridin-5-yl | 185.92 |
| 146 | 4-fluoro-3-methylcarbonylaminophenyl | 41.5 |

### Example 21

### Apomorphine Induced Deficits in Prepulse Inhibition of the Startle Response in Rats an in vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g. the reflex activity of the rats is measured. The reflex of the rats to the burst of noise may be attenuated by preceding the startle stimulus with a stimulus of lower intensity, at 3 to 12 db above background (65 db), which will attenuate the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine will reduce the inhibition of the startle reflex produced by the prepulse. Antipsychotic drugs such as haloperidol will prevent apomorphine from reducing the prepulse inhibition of the startle reflex. This assay may be used to test the antipsychotic efficacy of PDE10 inhibitors. Representative compounds provided herein were tested and determined to reduce the apomorphine-induced deficit in the prepulse inhibition of startle.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A compound of Formula (I): 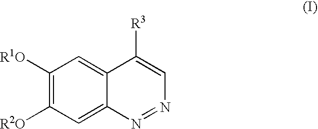
or an individual stereoisomer, a mixture of stereoisomers, or a pharmaceutically acceptable salt thereof wherein:
R¹ and R² are independently selected from hydrogen, alkyl, or haloalkyl; and
R³ is selected from formula (a)-(g): 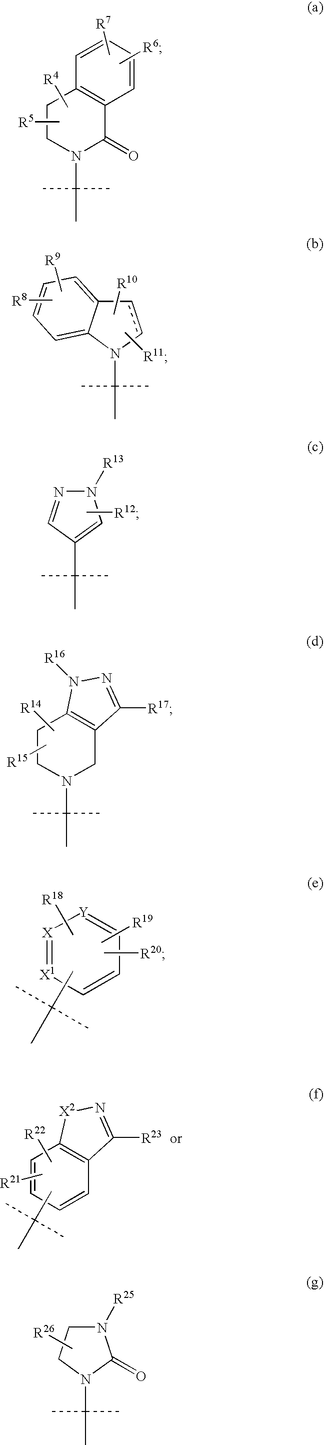
where:
X, X¹, and Y are all carbon; or one of X, X¹ and Y is carbon and the others are nitrogen; or two of X, X¹ and Y are carbon and the other is nitrogen;
X² is —NR²⁴—, —O—, or —S—;
dashed line in group (b) is an optional double bond;
each R⁴, R⁵, R¹⁰, R¹¹, R¹⁴, and R¹⁵ is independently hydrogen or alkyl; or any R⁴ and R⁵, R¹⁰ and R¹¹, or R¹⁴ and R¹⁵ form an oxo (═O) group;
each R¹⁸, R² , and R²² is independently hydrogen, alkyl, or halo; and
each R⁶, R⁷, R⁸, R⁹, R¹²R¹³, R¹⁶, R¹⁷, R¹⁹, R²⁰, R²³,R²⁴, R²⁵ and R²⁶ is independently hydrogen, alkyl, halo, haloalkyl, alkoxy, haloalkoxy, cyano, amino, monsubstituted or disubstituted amino, cycloalkylalkyl, aryl, heteroaryl, heterocyclyl, aralkyl, heteroaralkyl, heterocyclylalkyl, or —X³R²⁷ (where X³ is —O—, —CO—, —OC(O)—, —C(O)O, —NR²⁸CO—, —CONR²⁹—, —S—, —SO—, —SO₂—, —NR³⁰SO₂—, or —SO₂NR³¹— where R²⁸, R²⁹, R³⁰ and R³¹ are independently hydrogen, alkyl, hydroxyalkyl, alkoxyalkyl, aryl, aralkyl, heteroaryl, heteroaralkyl, acyl, or heterocyclylalkyl and R²⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl);
and wherein the aromatic or alicyclic ring in R⁶, R⁷, R⁸, R⁹, R¹², R¹³, R¹⁶, R¹⁷, R¹⁹, R²⁰, R²³, R²⁴, R²⁵, R²⁶, and R²⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, and optionally substituted heterocyclyl; and additionally substituted with one or two substitutents independently selected from R^{d} and R^{e} where R^{d} and R^{e} are hydrogen and fluoro;
provided that R⁶, R⁷, R⁸, R⁹, R¹², R¹³, R¹⁶, R¹⁷, R¹⁹, R²⁰, R²³, R²⁴, R²⁵ and R²⁶ are not independently selected from hydrogen, alkyl, halo, cyano, haloalkyl, alkoxy, haloalkoxy, and amino; and
provided that the compound of Formula (I) is not:
7-(cyclopropylmethoxy)-2-(6,7-dimethoxycinnolin-4-yl)-6-methoxy-3,4-dihydro-isoquinolin-1(2H)-one;
6-(cyclopropylmethoxy)-7-(6,7-dimethoxycinnolin-4-yl)-6-methoxy-3,4-dihydro-isoquinolin-1(2H)-one;
1-(6,7-dimethoxycinnolin-4-yl)-N-ethylindoline-5-sulfonamide;
1-(6,7-dimethoxycinnolin-4-yl)-N,N-diethylindoline-5-sulfonamide;
1-(6,7-dimethoxycinnolin-4-yl)-N-(2-propyl)indoline-5-sulfonamide;
N-(cyclopropylmethyl)-1-(6,7-dimethoxycinnolin-4-yl)indoline-5-sulfonamide;
N-(methyl)-1-(6,7-dimethoxycinnolin-4-yl)indoline-5-sulfonamide;
6,7-dimethoxy-4-(5-(methylsulfonyl)indolin-1-yl)cinnoline;
4-(5-(furan-3-yl)indolin-1-yl)-6,7-dimethoxycinnoline;
1-(6,7-dimethoxycinnolin-4-yl)-N-methylindoline-5-sulfonamide;
1-(6,7-dimethoxycinnolin-4-yl)-N,N-dimethylindoline-5-sulfonamide;
4-(1-benzyl-1H-pyrazol-4-yl)-6,7-dimethoxycinnoline;
6,7-dimethoxy-4-(5-(thiophen-3-yl)-2,3-dihydro-1H-indolin-1-yl)cinnoline;
6,7-dimethoxy-4-(5-(pyrimidin-5-yl)indolin-1-yl)cinnoline;
1-(6,7-dimethoxycinnolin-4-yl)-N,N-diisopropylindoline-5-sulfonamide;
1-(6,7-dimethoxycinnolin-4-yl)-N-(2-morpholinoethyl)indoline-5-sulfonamide;
N-cyclopropyl-1-(6,7-dimethoxycinnolin-4-yl)indoline-5-sulfonamide;
6,7-dimethoxy-4-(5-(pyrrolidin-1-ylsulfonyl)-2,3-dihydro-1H-indolin-1-yl)cinnoline;
1-(6,7-dimethoxycinnolin-4-yl)-N-(2-methoxyethyl)indoline-5-sulfonamide;
6,7-dimethoxy-4-(5-(pyridin-4-yl)indolin-1-yl)cinnoline;
4-(5-(3,5-dimethylisoxazol-4-yl)indolin-1-yl)-6,7-dimethoxycinnoline;
6,7-dimethoxy-4-(5-(piperidin-1-ylsulfonyl)indolin-1-yl)cinnoline;
3-(6,7-dimethoxycinnolin-4-yl)-N-ethylbenzamide;
N-cyclopropyl-3-(6,7-dimethoxycinnolin-4-yl)benzamide;
3-(6,7-dimethoxycinnolin-4-yl)-N,N-diethylbenzamide;
3-(6,7-dimethoxycinnolin-4-yl)-N-isobutylbenzamide;
6,7-dimethoxy-4-(5-(piperidin-1-ylcarbonyl)indolin-1-yl)cinnoline;
6-(benzyloxy)-2-(6,7-dimethoxycinnolin-4-yl)-3,4-dihydroisoquinolin-1(2H)-one;
N-cyclohexyl-3-(6,7-dimethoxycinnolin-4-yl)benzamide;
7-(cyclopropylmethoxy)-2-(6,7-dimethoxycinnolin-4-yl)-6-methoxy-3,4-dihydro-isoquinolin-1(2H)-one;
6-(cyclopropylmethoxy)-2-(6,7-dimethoxycinnolin-4-yl)-7-methoxy-3,4-dihydro-isoquinolin-1(2H)-one; and
2-(6,7-dimethoxycinnolin-4-yl)-5-(2-methoxyethoxy)-3,4-dihydroisoquinolin-1(2H)- one; or a pharmaceutically acceptable salt thereof.

2. The compound of claim 1 wherein R¹ and R² are alkyl and R³ is a group of formula (a).

3. The compound of claim 1 wherein R¹ and R² are alkyl and R³ is a group of formula:

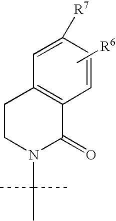

4. The compound of claim 3 wherein R⁷ is aryl, heteroaryl, or heterocyclyl optionally substituted with one to three substitutents independently selected from R^{a}, R^{b}, and R^{c}.

5. The compound of claim 1 wherein R³is a group of formula (b).

6. The compound of claim 1 wherein R³ is a group of formula

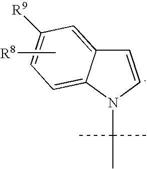

7. The compound of claim 1 wherein R³is a group of formula

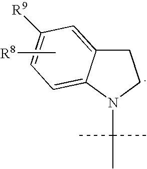

8. The compound of claim 1 wherein R³ is a group of formula

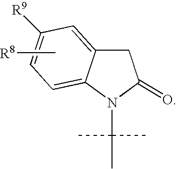

9. The compound of claim 1 wherein R³ is a group of formula

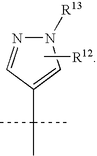

10. The compound of claim 9 where R¹³ is aralkyl optionally substituted with one to three substitutents independently selected from R^{a}, R^{b}, and R^{c} provided one of R^{a}, R^{b}, and R^{c} is other than hydrogen.

11. The compound of claim 1 wherein R³is a group of formula

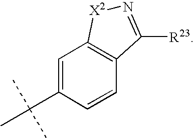

12. The compound claim 1 wherein R³ is a group of formula 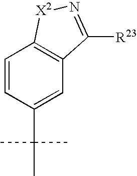
where X² is —S—.

13. The compound of claim 1 wherein R³ is a group of formula

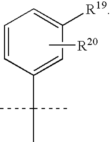

14. The compound of claim 1 wherein R³ is a group of formula: 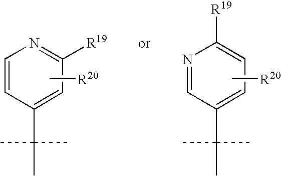
where R¹⁹ is phenyl optionally substituted with one to three substitutents independently selected from R^{a}, R^{b}, and R^{c}.

15. The compound of claim 1 wherein R³is a group of formula: 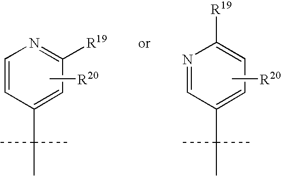
where R¹⁹ is heteroaryl optionally substituted with one to three substitutents independently selected from R^{a}, R^{b}, and R^{c}.

16. The compound of claim 1 wherein R³ is a group of formula: 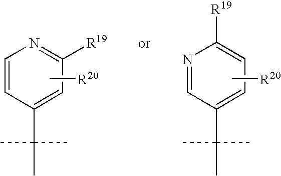
where R¹⁹ is heterocyclyl optionally substituted with one to three substitutents independently selected from R^{a}, R^{b}, and R^{c}.

17. The compound of claim 16 wherein R¹⁹ is piperidinyl, morpholinyl, or piperazinyl optionally substituted with one to three substitutents independently selected from R^{a}, R^{b}, and R^{c} provided that at least one of R^{a}, R^{b}, and R^{c} is other than hydrogen.

18. The compound of claim 1 wherein wherein R³ is a group of formula: 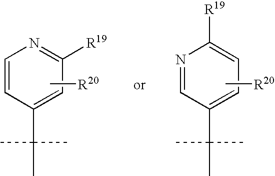
where R¹⁹ is monosubstituted or disubstituted amino.

19. A compound selected from Table 1 below:
TABLE 1

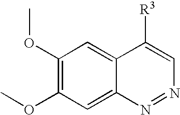

| Cpd | |
|---|---|
| # | R³ |
| 1 | 3-morpholin-4-ylphenyl |
| 2 | 4-piperidin-1-ylphenyl |
| 3 | 3-ethylsulfonylphenyl |
| 4 | 6-(piperidin-1-yl)-3,4-dihydroisoquinolin-1(2H)-one |
| 5 | 3-(1-methyl-1H-pyrazol-4-yl)phenyl |
| 6 | 1-phenyl-1H-pyrazol-4-yl |
| 7 | 3-(cyclopropylaminocarbonyl)-4,5,6,7-tetrahydro-1H-pyrazolo |
| | [4,3-c]-pyridin-5-yl |
| 8 | 3-(4,4-dimethyl-4,5-dihydro 1,3-oxazol-2-yl)phenyl |
| 9 | 3-(4,4-dimethyl-4,5-dihydro-1,3-oxazol-2-yl)pyridin-5-yl |
| 10 | 1-(3-methoxyphenyl)-1H-pyrazol-4-yl |
| 11 | 1-(3-ethoxyphenyl)-1H-pyrazol-4-yl |
| 12 | 3-(ethoxycarbonyl)-1H-indazol-5-yl |
| 13 | 3-(ethoxycarbonyl)-1H-indazol-6-yl |
| 14 | 3-acetylaminophenyl |
| 15 | 3-dimethylaminophenyl |
| 16 | 3-(thien-3-yl)phenyl |
| 17 | 3-(furan-3-yl)phenyl |
| 18 | 3-(4,4-dimethyl-4,5-dihydro-1,3-thiazol-2-yl)phenyl |
| 19 | 3-(cyclopropylaminocarbonyl)-1H-indazol-6-yl |
| 20 | 5-(morpholin-4-yl)indol-1-yl |
| 21 | 1-(4-methylbenzyl)-1H-pyrazol-4-yl |
| 22 | 1-(4-tert-butylbenzyl)-1H-pyrazol-4-yl |
| 23 | 1-(4-phenylbenzyl)-1H-pyrazol-4-yl |
| 24 | 1-(4-methoxycarbonylbenzyl)-1H-pyrazol-4-yl |
| 25 | 1-(2-phenylbenzyl)-1H-pyrazol-4-yl |
| 26 | 1-(3-trifluoromethylbenzyl)-1H-pyrazol-4-yl |
| 27 | 1-(2-trifluoromethylbenzyl)-1H-pyrazol-4-yl |
| 28 | 1-(2-cyanobenzyl)-1H-pyrazol-4-yl |
| 29 | 1-(3-methylbenzyl)-1H-pyrazol-4-yl |
| 30 | 1-(4-cyanobenzyl)-1H-pyrazol-4-yl |
| 31 | 1-(2-methylbenzyl)-1H-pyrazol-4-yl |
| 32 | 1-(4-trifluoromethoxybenzyl)pyrazol-4-yl |
| 33 | 6-(morpholin-4-yl)pyridin-3-yl |
| 34 | 2-(4-methylpiperazin-1-yl)pyridin-4-yl |
| 35 | 1-(4-fluorobenzyl)-1H-pyrazol-4-yl |
| 36 | 3-fluoro-2-(isopropylamino)pyridin-5-yl |
| 37 | 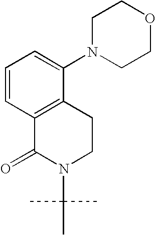 |
| | 5-morpholino-3,4-dihydroisoquinolin-(2H)-one |
| 38 | 3-cyclopropylaminocarbonylbenzo[d]isothiazol-5-yl |
| 39 | 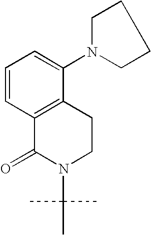 |
| 40 | 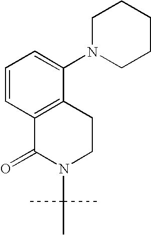 |
| 41 | 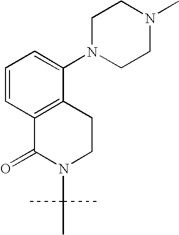 |
| 42 | 3-fluoro-2-morpholin-4-ylpyridin-4-yl |
| 43 | 2-(1-methylpiperazin-4-yl)pyrimidin-5-yl |
| 44 | 1-(2-fluorobenzyl)-1H-pyrazol-4-yl |
| 45 | 2-(piperidin-1-yl)pyridin-5-yl |
| 46 | 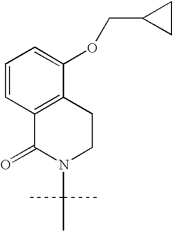 |
| 47 | 2-(1-methylpiperazin-4-yl)pyridin-5-yl |
| 48 | 3-cyclopropylaminocarbonylbenzo[d]isothiazol-6-yl |
| 49 | 3-cyclopropylaminocarbonylbenzo[d]isothiazol-7-yl |
| 50 | 2-(piperazin-1-yl)pyridin-4-yl |
| 51 | 2-(piperazin-1-yl)pyridin-5-yl |
| 52 | 3-ethoxycarbonylbenzo[d]isoxazol-5-yl |
| 53 | 3-ethoxycarbonylbenzo[d]isoxazol-6-yl |
| 54 | 2-(2-oxo-1-methylpiperazin-4-yl)pyridin-4-yl |
| 55 | 2-(3-methoxypyrrolidin-1-yl)pyridin-5-yl |
| 56 | 2-(4,4-difluoropiperidin-1-yl)pyridin-5-yl |
| 57 | 2-(3S-methylmorpholin-4-yl)pyridin-5-yl |
| 58 | 2-(3-methoxypiperidin-1-yl)pyridin-4-yl |
| 59 | 2-(isopropylamino)pyridin-5-yl |
| 60 | 2-(2-methylpropylamino)pyridin-5-yl |
| 61 | 2-[—NHCH(CH₃)CH₂CH₃]pyridin-5-yl |
| 62 | 2-(2-methoxyethylamino)pyridin-5-yl |
| 63 | 2-(2-aminoethylamino)pyridin-5-yl |
| 64 | 2-(pyrrolidin-1-yl)pyridin-5-yl |
| 65 | 2-(1-tert-butoxycarbonylazetidin-4-ylmethylamino)pyridin-5-yl |
| 66 | 2-(ethyl-n-propylamino)pyridin-5-yl |
| 67 | 2-(2R,6S-dimethylmorpholin-4-yl)pyridin-5-yl |
| 68 | 4-(quinolin-2-ylmethyloxy)phenyl |
| 69 | 2-(4-methoxypiperidin-1-yl)pyridin-5-yl |
| 70 | 2-(2-oxo-1-methylpiperazin-1-yl)pyridin-5-yl |
| 71 | 2-(4-fluoropiperidin-1-yl)pyridin-5-yl |
| 72 | 2-(3-(trifluoromethyl)-5,6,7,8-tetrahydro-[1,2,4]triazolo[4,3-a] |
| | pyrazin-7-yl)pyridin-5-yl |
| 73 | 2-(methylisopropylamino)pyridin-5-yl |
| 74 | 2-(4-methoxyazetidin-1-yl)pyridin-5-yl |
| 75 | 3-benzyloxyphenyl |
| 76 | 2-(4-oxopiperidin-1-yl)pyridin-5-yl |
| 77 | 2-[—N(CH₃)(CH₂CH(CH₃)₂)]pyridin-5-yl |
| 78 | 2-(4,4-difluoroazetidin-1-yl)pyridin-5-yl |
| 79 | 2-(4-methylaminopiperidin-1-yl)pyridin-5-yl |
| 80 | 3-diethylaminocarbonylbenzo[d]isothiazol-6-yl |
| 81 | 2-(phenylamino)pyridin-5-yl |
| 82 | 2-(oxazol-2-yl)phenyl |
| 83 | 3-isopropylaminocarbonylbenzo[d]isothiazol-6-yl |
| 84 | 2-(2-tert-butoxyethylamino)pyridin-5-yl |
| 85 | 2-(S—NHCH(CH₃)CH₂OCH₃)pyridin-5-yl |
| 86 | 2-[(methyl)-(2-tert-butylethylamino)amino]pyridin-5-yl |
| 87 | 2-(phenoxy)pyridin-5-yl |
| 88 | 2-(4-dimethylaminophenylamino)pyridin-5-yl |
| 89 | 2-(cyclopropylmethyloxy)pyridin-5-yl |
| 90 | 2-(1-tert-butoxycarbonyl-1,2,5,6-tetrahydropyridin-4-yl)pyridin-5- |
| | yl |
| 91 | 2-(2-ethoxyethylamino)pyridin-5-yl |
| 92 | 2-[(isopropyl)-(2-tert-butylaminoethyl)aminopyridin-5-yl |
| 93 | 2-(2-pyridin-2-ylethylamino)pyridin-5-yl |
| 94 | 2-(dimethylamino)pyridin-5-yl |
| 95 | 2-(3-cyanophenylamino)pyridin-5-yl |
| 96 | 2-(1,2,5,6-tetrahydropyridin-4-yl)pyridin-5-yl |
| 97 | 2-(2-bromo-4,5,6,7-tetrahydrothiazolo[5,4-c]pyridin-1-yl)pyridin-5- |
| | yl |
| 98 | 2-(3-fluorobenzylamino)pyridin-5-yl•TFA |
| 99 | 2-(thiophen-2-ylmethylamino)pyridin-5-yl |
| 100 | 3-fluoro-4-acetylphenyl |
| 101 | 4-(4-carboxyazetidin-1-yl)phenyl |
| 102 | 2-(pyridin-3-ylmethylamino)pyridin-5-yl |
| 103 | 2-(3-tert-butylphenylamino)pyridin-5-yl |
| 104 | 2-(1-methylbenzylamino)pyridin-5-yl |
| 105 | 2-(2,2-dimethylpropylamino)pyridin-5-yl |
| 106 | 2-(1-methyl-1,2,5,6-tetrahydropyridin-4-yl)pyridin-5-yl |
| 107 | 2-(n-butylamino)pyridin-5-yl |
| 108 | 2-(4-hydroxy-4-phenylpiperidin-1-yl)pyridin-5-yl |
| 109 | 2-(4-carboxyazetidin-1-yl)pyridin-5-yl |
| 110 | 2-(3R,5S-dimethylpiperazin-1-yl)pyridin-5-yl |
| 111 | 2-[methyl-(2-pyridin-2-ylethyl)amino]pyridin-5-yl |
| 112 | 2-[methyl-(2-phenylethyl)amino]pyridin-5-yl |
| 113 | 2-(2-methylbenzo[d]thiazol-6-ylamino)pyridin-5yl |
| 114 | 2-(2-hydroxy-2-methylpropylamino)pyridin-5-yl |
| 115 | 3-fluoro-2-[3-fluoro-2-(isopropylamino)pyridin-5-yl]pyridin-5-yl |
| 116 | 2-(4-cyano-4-phenylpiperidin-1-yl)pyridin-5-yl |
| 117 | 2-(2-amino-2-methylpropylamino)pyridin-5-yl |
| 118 | 2-(4-(oxazol-5-yl)phenylamino)pyridin-5-yl |
| 119 | 2-[5-(cyclopropyl)-[1.3.4]-thiadiazol-2-ylamino]pyridin-5-yl |
| 120 | 2-(2-indol-3-ylethylamino)pyridin-5-yl |
| 121 | 2-(benzothiophen-2-ylmethylamino)pyridin-5-yl |
| 122 | 2-(3-trifluoromethoxybenzylamino)pyridin-5-yl |
| 123 | 6-isopropylamino-2-methylpyridin-3-yl |
| 124 | 2-(2-pyridin-4-ylethylamino)pyridin-5-yl |
| 125 | 2-(2-pyridin-3-ylethylamino)pyridin-5-yl |
| 126 | 2-(1S-methylbenzylamino)pyridin-5-yl |
| 127 | 2-(1R-methylbenzylamino)pyridin-5-yl |
| 128 | 2-(1RS-methylbenzylamino)pyridin-5-yl |
| 129 | 2-(2-phenylethylamino)pyridin-5-yl |
| 130 | 2-[2-(2-methoxyphenylethyl)amino]pyridin-5-yl |
| 131 | 2-(5-methylfuran-2-ylmethylamino)pyridin-5-yl |
| 132 | 2-(pyridin-2-ylmethylamino)pyridin-5-yl |
| 133 | 2-[2-(3-methoxyphenyl)ethylamino]pyridin-5- |
| 134 | 2-(2-phenylpropylamino)pyridin-5-yl |
| 135 | 3-(morpholin-4-yl)phenyl |
| 136 | 4-(piperidinyl-1-yl)phenyl |
| 137 | 2-[N-(2-tert-butylaminoethyl)-N-methylamino]pyridin-5-yl |
| 138 | 2-(4-dimethylaminophenylamino)pyridin-5-yl |
| 139 | 2-[N-(2-tert-butylaminoethyl)-N-(2-propyl)amino]pyridin-5-yl |
| 140 | 2-(4-oxazol-5-ylphenylamino)pyridin-5-yl |
| 141 | 2-(5-cyclopropyl-[1.3.4]-thiazol-2-ylamino)pyridin-5-yl |
| 142 | 2-(3-trifluoromethoxybenzylamino)pyridin-5-yl |
| 143 | 2-[1-(4-fluorophenyl)ethylamino)pyridin-5-yl |
| 144 | 2-[2-(3-methoxyphenyl)ethylamino]pyridin-5-yl |
| 145 | 2-(2-phenylpropylamino)pyridin-5-yl |
| 146 | 4-fluoro-3-methylcarbonylaminophenyl |
| 147 | 2-[1-(tert-butyloxycarbonyl)pyrrolidin-3S-yl]pyridin-5-yl |
| 148 | 4-(4-hydroxypiperidin-1-yl)pyridin-5-yl |
| 149 | 3-methyl-2-(2-isopropylamino)pyridin-5-yl |
| 150 | 2-(pyridin-4-ylmethylamino)pyridin-5-yl |
| 151 | 2-[1-(pyridin-2-ylmethyl)ethylamino)pyridin-5-yl |

or a pharmaceutically acceptable salt thereof.

20. A pharmaceutical composition comprising a compound of claim 1 and a pharmaceutically acceptable expicient.

21. A method of treating a disorder treatable by inhibition of PDE10 enzyme in a patient which method comprises administering to the patient a pharmaceutical composition comprising a compound of claim 1 and a pharmaceutically acceptable expicient.

22. The method of claim 21 wherein the disease is schizophrenia, bipolar disorder, or obsessive-compulsive disorder.
